SEQUENCE LISTING (23 sequences)

SEQ ID NO: 1 - protein, 56 aa, synthetic construct
VNQHLCGSDLVEALYLVCGERGFFYTPETEEGPRRGIVEQCCHSICSLEQLENYCN

SEQ ID NO: 2 - protein, 60 aa, Lymphocystis disease virus 4
PLCGSELVDALELVCHDRGGFYSPTIKEVHTNRKTQSGIVEKCCRKEGCDYAYLTQYCAH

SEQ ID NO: 3 - protein, 58 aa, Singapore grouper iridovirus
QLQVCGGELIDALTEHCGDRGVYTPPRRGRRTRSVGLADACCKNECDENELDRYCNPK

SEQ ID NO: 4 - protein, 31 aa, synthetic construct
SLEEEWAQIECEVYGRGCPKPDFYDWFERQL

SEQ ID NO: 5 - protein, 50 aa, synthetic construct
GSDLVEALYLVLGERGFFYTDPTGGGPRRGIVEQLLHSILSLYQLENYLN

SEQ ID NO: 6 - protein, 57 aa, synthetic construct
FVNQHLCGSDLVEALYLVCGERGFFYTDPTGGQPRRGIVEQCCHSICSLYQLENYCN

SEQ ID NO: 7 - protein, 67 aa, synthetic construct
MPSVQEVEKLLHVLDRNGDGKVSAEELKAFADDSKCPLDSNKIKAFIKEHDKNKDGKLDLKELVSIL

SEQ ID NO: 8 - protein, 97 aa, synthetic construct
HHHHHHMPSVQEVEKLLHVLDRNGDGKVSAEELKAFADDSKCPLDSNKIKAFIKEHDKNKDGKLDLKELVSILSSSSSSSNNNNNNNNNNENLYFQG

SEQ ID NO: 9 - protein, 7 aa, synthetic construct
ENLYFQG

SEQ ID NO: 10 - protein, 155 aa, synthetic construct
MHHHHHHMPSVQEVEKLLHVLDRNGDGKVSAEELKAFADDSKCPLDSNKIKAFIKEHDKNKDGKLDLKELVSILSSSSSSSNNNNNNNNNNENLYFQGFVNQHLCGSDLVEALYLVCGERGFFYTPETEEGPRRGIVEQCCHSICSLEQLENYCN

SEQ ID NO: 11 - protein, 160 aa, synthetic construct
MGHHHHHHMPSVQEVEKLLHVLDRNGDGKVSAEELKAFADDSKCPLDSNKIKAFIKEHDKNKDGKLDLKELVSILSSSSSSSNNNNNNNNNNENLYFQGMPLCGSELVDALELVCHDRGGFYSPTIKEVHTNRKTQSGIVEKCCRKEGCDYAYLTQYCAH

SEQ ID NO: 12 - protein, 158 aa, synthetic construct
MGHHHHHHMPSVQEVEKLLHVLDRNGDGKVSAEELKAFADDSKCPLDSNKIKAFIKEHDKNKDGKLDLKELVSILSSSSSSSNNNNNNNNNNENLYFQGMQLQVCGGELIDALTEHCGDRGVYTPPRRGRRTRSVGLADACCKNECDENELDRYCNPK

SEQ ID NO: 13 - protein, 131 aa, synthetic construct
MGHHHHHHMPSVQEVEKLLHVLDRNGDGKVSAEELKAFADDSKCPLDSNKIKAFIKEHDKNKDGKLDLKELVSILSSSSSSSNNNNNNNNNNENLYFQGMSLEEEWAQIECEVYGRGCPKPDFYDWFERQL

SEQ ID NO: 14 - protein, 150 aa, synthetic construct
MGHHHHHHMPSVQEVEKLLHVLDRNGDGKVSAEELKAFADDSKCPLDSNKIKAFIKEHDKNKDGKLDLKELVSILSSSSSSSNNNNNNNNNNENLYFQGMGSDLVEALYLVLGERGFFYTDPTGGGPRRGIVEQLLHSILSLYQLENYLN

SEQ ID NO: 15 - protein, 157 aa, synthetic construct
MGHHHHHHMPSVQEVEKLLHVLDRNGDGKVSAEELKAFADDSKCPLDSNKIKAFIKEHDKNKDGKLDLKELVSILSSSSSSSNNNNNNNNNNENLYFQGMFVNQHLCGSDLVEALYLVCGERGFFYTDPTGGQPRRGIVEQCCHSICSLYQLENYCN

SEQ ID NO: 16 - protein, 153 aa, synthetic construct
MGHHHHHHMPSVQEVEKLLHVLDRNGDGKVSAEELKAFADDSKCPLDSNKIKAFIKEHDKNKDGKLDLKELVSILSSSSSSSNNNNNNNNNNENLYFQGMHLCGEDLEEALKLVCGERGFYYVDPTDGNPENTVVKRCCEDVCSVEQLERFCN

SEQ ID NO: 17 - protein, 158 aa, synthetic construct
MGHHHHHHMPSVQEVEKLLHVLDRNGDGKVSAEELKAFADDSKCPLDSNKIKAFIKEHDKNKDGKLDLKELVSILSSSSSSSNNNNNNNNNNENLYFQGMQIQVCGGELIETLQRFCGDRGVWFPPNNDQDDRDKDLFDACCKNECNENELDRYCNPK

SEQ ID NO: 18 - protein, 157 aa, synthetic construct
MGHHHHHHMPSVQEVEKLLHVLDRNGDGKVSAEELKAFADDSKCPLDSNKIKAFIKEHDKNKDGKLDLKELVSILSSSSSSSNNNNNNNNNNENLYFQGMFVNQHYCGSDLVEALYLVCGERGFIYYDPTNNQPRRSIWEQCCHNICSLEQLENYCN

SEQ ID NO: 19 - protein, 153 aa, synthetic construct
MGHHHHHHMPSVQEVEKLLHVLDRNGDGKVSAEELKAFADDSKCPLDSNKIKAFIKEHDKNKDGKLDLKELVSILSSSSSSSNNNNNNNNNNENLYFQGMHLCGEDLEDALRLVCGERGFYFVDETDGNPQNTVLRRCCNDVCSVEQLERFCN

SEQ ID NO: 20 - DNA, 468 bp, synthetic construct
  source 1..468: mol_type = other DNA
atgcaccaccaccaccaccacatgccgagcgtgcaggaagtggaaaaactgctgcacgtgctggaccgtaacggtgatggcaaagttagcgcggaggaactgaaagcgttcgcggacgatagcaagtgcccgctggatagcaacaagatcaaagcgtttattaaggaacacgacaagaacaaagatggcaagctggacctgaaagagctggttagcatcctgagcagcagcagcagcagcagcaataacaataataacaacaacaacaacaacgaaaacctgtacttccagggttttgttaaccaacatctgtgcggcagcgacctggtggaggcgctgtacctggtttgcggtgaacgtggcttcttttataccccggagaccgaggaaggtccgcgtcgtggcatcgtggagcagtgctgccacagcatttgcagcctggagcaactggaaaactattgcaactaa

SEQ ID NO: 21 - DNA, 483 bp, synthetic construct
  source 1..483: mol_type = other DNA
atgggacatcatcaccaccatcatatgccgtcagtgcaagaagtggaaaaactcttacatgtattagatcgtaatggtgatggcaaggtttcggctgaagagctgaaagcattcgccgacgatagcaaatgccccctcgatagtaataaaattaaagccttcataaaagagcatgacaaaaataaagatggaaaattggacctgaaggaacttgtatctatattgtcatcatcatcctcgagttcaaataacaacaataataacaataataataacgaaaacctctactttcagggcatgccgctgtgcgggagcgagcttgttgatgcactggaactggtttgtcacgatcgcggaggcttttactctcctacaatcaaggaggttcatacaaaccgtaaaacgcaatcaggtatcgtggagaaatgctgccgcaaagagggctgtgattacgcgtatttaactcagtactgtgcacactaa

SEQ ID NO: 22 - DNA, 477 bp, synthetic construct
  source 1..477: mol_type = other DNA
atgggacaccatcatcaccatcatatgccgtcagttcaagaggtcgaaaagttgttgcatgtacttgatcgtaacggcgacggaaaagtgagcgccgaggagttaaaagcattcgcagacgatagtaaatgcccattagatagcaacaaaatcaaggctttcataaaagagcatgataagaataaggatggtaagcttgaccttaaagagctggtgagtatactctctagcagcagtagctcttccaacaataacaacaataacaataacaataatgagaatttgtacttccagggtatgcaacttcaggtttgcggtggcgaactcatagacgcgttaacagaacactgtggagatcggggtgtttacactccacctcgtagagggcgtagaactcgctctgttggcttagcggacgcttgctgtaagaacgagtgtgacgaaaacgaactcgacagatactgtaaccccaagtaa

SEQ ID NO: 23 - DNA, 396 bp, synthetic construct
  source 1..396: mol_type = other DNA
atgggacaccatcatcatcatcacatgcctagcgtgcaagaagtggagaaactgctgcacgtattggatcgaaacggcgatggtaaagtgtcagcggaggaattgaaagcattcgcggacgacagtaagtgtcccctggacagcaacaagatcaaagcttttataaaggagcatgacaaaaataaagatggaaaactggacttgaaagagttggtcagtattctgtctagtagtagctcatcaagtaacaataacaacaataacaacaataataacgaaaacctgtattttcagggcatgagtctggaagaagagtgggcacagatagaatgcgaagtttatggacgtggttgcccgaaacctgatttttatgattggttcgaacgacaactctaa

(19) United States
(12) Patent Application Publication
(10) Pub. No.: US 2026/0297529 A1
(43) Pub. Date: Oct. 1, 2026

(54) GROWTH FACTOR SUBSTITUENTS

(71) Applicant: ALEPH FARMS LTD., Rehovot (IL)

(72) Inventors: Neta Lavon, Ness Ziona (IL); Ayelet Maor-Shoshani, Ness Ziona (IL); Gideon Lapidoth, Tel Aviv (IL); Lior Zimmerman, Rehovot (IL); Noga Alon, Yavne (IL); Itay Levin, Herzeliya (IL); Dror Baran, Tel Aviv (IL)

(21) Appl. No.: 19/480,406

(22) PCT Filed: May 8, 2024
(86) PCT No.: PCT/IL2024/050438

§ 371 Date: Oct. 31, 2025

Related U.S. Application Data
(60) Provisional application No. 63/500,921, filed on May 9, 2023.

(51) Int. Cl. C12N 5/077; C07K 14/00; C07K 14/005

(52) U.S. Cl. CPC C12N 5/0658; C07K 14/001; C07K 14/005; C12N 2501/998

(57) ABSTRACT

Growth-enhancing peptides and fusion polypeptide are provided. The peptides and fusion polypeptides may be used in cell culture, in particular in the production of cultured meat, as replacement or addition to insulin. There is further provided a cell culture medium comprising at least one of the peptides or fusion polypeptides, and a method for growing cells comprising adding at least one of the peptides or polypeptides to the cell culture medium.

### FIELD OF THE INVENTION

The present invention is in the fields of cell biology and cell culturing and relates to insulin substitutes effective in enhancing cell growth.

### BACKGROUND OF THE INVENTION

The increasing demands and expectations which are directed towards the biotechnology industry, produce an immense motivation to develop high performance biological processes, while minimizing the required time frame and reducing costs. Cell cultures are one of the most useful tools in life science research and biotechnology, irreplaceable for applications as diverse as testing of drugs or toxins, development of gene and cell therapies, investigation of the function of biomolecules, production of biologics or vaccine particles and more (Irina et al., 2022, Metabolomics Perspectives, pg. 415).

Recently, a new role of cell culture has been gaining increased momentum, namely, culturing of metazoan cells in vitro to produce cultured meat, as an alternative to traditional animal meat obtained from slaughtered animals (herein referred to as “animal-derived meat”). Such meat (also known and referred to interchangeably as cultured meat, cultivated meat, cell-based meat, in vitro produced cell-based meat, cell culture-based meat, in vitro meat, lab-grown meat, or clean meat) offers a sustainable alternative to traditional meat and overcomes animal-related drawbacks such as microbial contamination and possible exposure to hormones and drugs, such as antibiotics. Replacing animal-derived meat with cultured meat is expected to provide substantial environmental benefits, such as conservation of land and water and reduction of greenhouse gas emissions. Furthermore, increasing concerns regarding animal welfare are also addressed by the prospect of the cultured meat alternative.

One of the main limiting factors in the context of cell culture is the rate of cell growth. While there is some variation across different cell types, doubling time (i.e., the time required to double the number of cells in a population) is typically between 12-24 hours. The conditions in which the cells are cultured significantly affects the overall cell performance and growth rate and is therefore a crucial consideration in cell cultivation.

In order to allow cell cultivation outside of a live organism, conditions which mimic physiological ones must be maintained. These conditions include, for example, a temperature of 37° C., a pH of about 7-7.5, and regulated concentrations of oxygen and CO₂. In addition, a variety of nutrients and other compounds must be provided within the cell media, which allow cell growth and proliferation, including carbon and nitrogen sources, salts, vitamins, and hormones. Serum, derived from animal sources such as fetal bovine, is a commonly used component of most cell culture media, providing the required nutrients and proteins for efficient cell culture. However, this source of nutrients suffers from several limitations. The composition of animal-derived serum tends to exhibit batch-to-batch variability and requires tight regulation against pathogens such as bacterial infection, or alternatively, against the presence of drugs such as antibiotics. Keeping up with the industrial demands of serum for cell culture is challenging, and there are growing concerns regarding animal welfare. The welfare consideration is especially acute in industries such as the cultured meat industry, the goal of which is to create substitutes for animal-derived products. On the other hand, cell cultures grown in serum-free media often require supplementation of nutritional components. Identifying the precise required components and their respective optimal concentrations is an ongoing target of much research, and independent supplying of each component is costly. The high costs of cell-culture medium, specifically serum-free medium, are of particular concern in the industry of cell-based meat production. Production of cell-based meat is intricately linked to the cell culture media used in its cultivation, and is currently estimated to account for about 80% of the cost of the end product.

One of the more costly components required for cell culture is the growth factor insulin. Insulin is a protein composed of 51 amino acids and has a molecular weight of 5.8 kD. It is a heterodimer of an A-chain and a B-chain, which are linked together by disulfide bonds. In vivo, insulin is secreted by the beta-cells of the pancreatic islets of Langerhans, in response to elevated glucose levels in the blood, and enhances sugar uptake and metabolism in the cells. Insulin has also been found to inhibit apoptosis caused by serum withdrawal in several types of cells. Insulin, and the related insulin-like growth factors IGF-I and IGF-II, act on cells through binding to specific receptors, the insulin receptor (IR) and the type 1 IGF receptor (IGF-1R), respectively, two highly homologous transmembrane glycoproteins belonging to the receptor tyrosine kinase (RTK) family. As suggested by their name, IGF-I and IGF-II are cell growth factors, participating in anti-apoptotic and mitogenic pathways. Although Insulin and IGF-I have high specificity for their cognate receptors, each of them can also bind the alternative receptor, at a 10²-10³ reduced affinity. There is much overlapping between the signaling pathways of IR and IGF-1R.

In mammalian cell culture, recombinant insulin is added as a growth factor at approximately 100 times the physiological concentration. Insulin at this high concentration produces a cell-growth enhancing effect, including inhibition of apoptosis and enhancement of mitosis (Benni and Patil, 2016, Basic and Clini. Physio, and Pharmac., 27(5), 445-456). These effects are not only through activation of the IR but also through activation of the IGF-1R.

Qing-xin et al., (Jou, of Bio. Chem., 2008, 283(21), 14703-14716) disclose a novel design, structure, and function of a single-chain insulin (SCI) analog. The single-chain insulin analog (SCI-57) contains the amino acid sequences of native chains A and B with several substitutions, and a 6-residue linker (GGGPRR) therebetween. The substitutions comprise one substitution in the A-domain (Thr^{A8} to His) and three in the B-domain: His^{B10} to Asp, Pro^{B28} to Asp, and Lys^{B29} to Pro. The thermodynamic stability of SCI-57 is increased, relative both to wild type (WT) insulin and to a corresponding two-chain insulin analog.

Glidden et al., (J. Biol. Chem., 2018, 293(1) 47-68), describe the structure, function, and stability of additional SCI analogs of 57-residues, based on the SCI-57 disclosed by Qing-xin, including several modifications. Cell-based studies of these SCI revealed native-like signaling properties with negligible mitogenic activity. The SCI crystal structure, determined as a zinc-free hexamer at 2.8 A°, revealed a native insulin fold with incomplete or absent electron density in the C domain. The stability of the analog was greater than that of WT insulin. Furthermore, the analog was found to be less prone to self-assembly and aggregation at high concentrations than the WT insulin.

The efficiency and cost of insulin for use in cell culture remains one of the main factors which limits the possibility of cultured meat becoming a significant substitute to natural animal-derived meat. There remains a need for lowering the costs of cultured-meat production, and particularly for increasing the efficiency and consequently decreasing the price of agents used in cell culture.

### SUMMARY OF THE INVENTION

The present invention provides peptides, polypeptides and fusions thereof, and their use as growth factors in cell culture. The present invention further provides cell-culture medium comprising peptides, polypeptides and fusions at low concentration relative to insulin concentration in cell culture, and methods for growing cells under low concentrations of growth factors. According to particular aspects of the present invention, the medium and methods are for culturing of meat.

The present invention is based in part on the unexpected finding that few peptides and fusion polypeptides selected from a large collection of potentially relevant sequences, with no previously known cell-growth enhancing activity, enhance cell growth in culture. Surprisingly, it has further been found that the cell-growth enhancing activity of the fusion polypeptides is achieved at low concentrations relative to the generally used concentrations of standard growth factors, such as insulin.

The present invention provides, according to one aspect, a fusion polypeptide, comprising a sequence selected from the group consisting of SEQ ID NO: 2, SEQ ID NO: 3, SEQ ID NO: 4, SEQ ID NO: 5, SEQ ID NO: 6, and an analog or derivative thereof. According to some embodiments, the analog or derivative has at least 90% identity to the selected sequence. The fusion polypeptide further comprises a heterologous sequence of 5-100 amino acid residues. According to some embodiments, the fusion polypeptide comprises a sequence selected from the group consisting of SEQ ID NO: 2, SEQ ID NO: 3, SEQ ID NO: 4, and an analog or derivative thereof. According to some embodiments, the analog or derivative has at least 90% identity to the selected sequence.

According to some embodiments, the analog comprises at least 95% identity to a sequence selected from the group consisting of SEQ ID NO: 2, SEQ ID NO: 3, and SEQ ID NO: 4. According to other embodiments, the analog comprises 1-5 substitutions, additions, or deletions of amino acid residues to a sequence selected from the group consisting of SEQ ID NO: 2, SEQ ID NO: 3, SEQ ID NO: 4.

According to some embodiments the heterologous sequence comprises at least one moiety selected from a solubility-enhancing peptide, a carrier polypeptide, and a permeability enhancing moiety. According to some embodiments the solubility-enhancing peptide comprises SEQ ID NO: 7, or an analog or derivative thereof. According to some embodiments, the analog or derivative has at least 90% identity to SEQ ID NO: 7. According to some embodiments, the heterologous sequence comprises at least one sequence selected from: a poly-histidine comprising 2-10 histidine residues, a linker of 2-30 amino acid residues, and a protease cleavage site. According to some particular embodiments, the linker comprises serine and asparagine residues. According to some particular embodiments, the linker consists of serine and asparagine residues. According to some embodiments, the protease cleavage site comprises SEQ ID NO: 9. According to further embodiments, the protease cleavage site consists of SEQ ID NO: 9. According to some embodiments the heterologous sequence comprises SEQ ID NO: 8, or an analog or derivative thereof. According to some embodiments, the analog or derivative has at least 90% identity to SEQ ID NO: 8. According to some embodiments the heterologous sequence comprises SEQ ID NO: 8, or an analog or derivative thereof having at least 95% identity. According to some embodiments the heterologous sequence consists of SEQ ID NO: 8.

According to some embodiments, the fusion polypeptide consists of a sequence selected from SEQ ID NO: 11, SEQ ID NO: 12, SEQ ID NO: 13, and an analog or derivative thereof. According to some embodiments, the analog or derivative has at least 90% identity to the selected sequence. According to some embodiments, the fusion polypeptide is an analog having at least 95% identity to a sequence selected from SEQ ID NO: 11, SEQ ID NO: 12, SEQ ID NO: 13. According to some embodiments, the fusion polypeptide is an analog having at least 98% identity to a sequence selected from SEQ ID NO: 11, SEQ ID NO: 12, SEQ ID NO: 13. According to some embodiments, the fusion polypeptide is an analog having 1-5 substitutions, additions, or deletions of amino acid residues to a sequence selected from SEQ ID NO: 11, SEQ ID NO: 12, SEQ ID NO: 13.

According to some embodiments, the fusion polypeptide is for use in enhancing cell growth in cell culture or tissue culture. According to some embodiments, the cell growth comprises at least one of enhancing cell proliferation, enhancing cell differentiation, and enhancing cell maturation.

According to some embodiments, the cell culture comprises cells which are grown in suspension culture. According to some further embodiments, the cell culture comprises cells which are grown on a scaffold.

According to some particular embodiments, the cell culture is used in the process of producing cultured meat. According to some embodiments the tissue culture is formulated to cultured meat.

According to some embodiments, the fusion polypeptide is for use in replacing all or part of the insulin supplement used as a growth factor in cell culture or tissue culture. According to some particular embodiments, the fusion polypeptide is for use in enhancing cell growth in cell culture, at a low concentration relative to insulin concentration in cell culture. According to some embodiments, the low concentration in cell culture comprises less than 0.5, 0.25, less than a 0.15, or less than 0.1 of the insulin molar concentration in cell culture. According to some embodiments, the fusion polypeptide for replacing insulin is provided at a weight concentration of about 0.1 μg/ml to 1 μg/ml. According to other embodiments, the fusion polypeptide for replacing insulin is provided at a molar concentration of about 5 nM to 200 nM.

The present invention provides, according to a further aspect, a polynucleotide encoding any fusion polypeptide disclosed above. According to some embodiments, the polynucleotide encodes a sequence selected from SEQ ID NO: 11, SEQ ID NO: 12, and SEQ ID NO: 13. According to some embodiments, the polynucleotide comprises a sequence selected from SEQ ID NO: 21, SEQ ID NO: 22, SEQ ID NO: 23, and an analog or derivative having at least 80% identity, at least 85% identity, at least 90% identity or at least 95% identity to the selected nucleic acid sequence. According to some embodiments, the polynucleotide is part of a vector, for example a recombinant vector or plasmid. According to further embodiments, a cell, or a population of cells, transfected with said polynucleotide or vector, is provided. According to some embodiments, the cell is a bacterial cell. According to some embodiments, the cell, or population of cells, transfected with said polynucleotide or vector is for production of a recombinant polypeptide.

The present invention provides, according to a further aspect, a cell-culture medium, comprising any fusion polypeptide as described above, or at least one peptide selected from the group consisting of SEQ ID NO: 2, SEQ ID NO: 3, SEQ ID NO: 4, and an analog or derivative thereof. According to some embodiments, the analog or derivative has at least 90% identity to the selected sequence. According to some embodiments, the cell-culture medium comprises at least one polypeptide selected from the group consisting of: SEQ ID NO: 11, SEQ ID NO: 12, and SEQ ID NO: 13. According to some embodiments, the cell-culture comprises about 0.1-1 μg/ml of a polypeptide selected from the group consisting of: SEQ ID NO: 11, SEQ ID NO: 12, and SEQ ID NO: 13. According to some embodiments, the cell-culture comprises about 0.25-0.5 μg/ml of a polypeptide selected from the group consisting of: SEQ ID NO: 11, SEQ ID NO: 12, and SEQ ID NO: 13. According to some embodiments, the cell-culture comprises a concentration of about 5 nM to 200 nM of a polypeptide selected from the group consisting of: SEQ ID NO: 11, SEQ ID NO: 12, and SEQ ID NO: 13. According to some embodiments, the cell-culture medium comprises at least one supplement selected from a natural colorant and cobalamin (vitamin B12). According to further embodiments, the cell-culture medium further comprises at least one supplement selected form the group consisting of iron or a salt thereof, folate, zinc or a salt thereof, selenium or a salt thereof, vitamin D, vitamin E, Coenzyme Q10, a fatty acid, and combinations thereof. According to some embodiments, the cell-culture medium is serum-free.

The present invention provides, according to a further aspect, a peptide comprising a sequence selected from SEQ ID NO: 2, SEQ ID NO: 3, SEQ ID NO: 4, an analog or derivative thereof or a fusion polypeptide thereof, for use as a growth-enhancing factor. According to some embodiments, the peptide or fusion polypeptide consists of an amino acid selected from SEQ ID NO: 2, SEQ ID NO: 3, SEQ ID NO: 4, and additional 1-50, 2-30 or 5-20 amino acid residues. According to additional embodiments, the peptide or fusion polypeptide comprises at least one non-proteinaceous moiety, including but not limited to carbohydrates, lipids, metals etc. According to some embodiments, the analog or derivative has at least 90% identity to the selected sequence. According to some embodiments, the peptide is for use in enhancing cell growth in a cell culture. According to some embodiments the cell culture is used in the process of producing cultured meat. According to some embodiments, the peptide promotes cell growth at a low concentration in cell culture relative to insulin concentration in cell culture. According to some embodiments the low concentration in cell culture is a low molar concentration, and comprises less than 0.25, less than a 0.15, or less than 0.1 of the insulin molar concentration in cell culture. According to some embodiments, the peptide promotes cell growth at a weight concentration of about 0.1 μg/ml to 1 μg/ml. According to other embodiments, the peptide promotes cell growth at a molar concentration of about 5 nM to 200 nM.

The present invention provides, according to a further aspect, a method for culturing cells, the method comprising supplementing a growth medium with at least one peptide or fusion polypeptide as described above, and adding cells to the growth medium. According to some embodiments, the peptide or polypeptide comprise a sequence selected from the group consisting of SEQ ID NO: 2, SEQ ID NO: 3, SEQ ID NO: 4, SEQ ID NO: 5, SEQ ID NO: 6, and an analog or derivative thereof. According to some embodiments, the analog or derivative has at least 90% identity to the selected sequence. According to some particular embodiments, the peptide or polypeptide comprise at least one sequence selected from the group consisting of SEQ ID NO: 2, SEQ ID NO: 3, SEQ ID NO: 4, and an analog or derivative thereof. According to some embodiments, the analog or derivative has at least 90% identity to the selected sequence. According to some embodiments, the peptide or polypeptide comprise at least one sequence selected from the group consisting of SEQ ID NO: 10, SEQ ID NO: 11, SEQ ID NO: 12, SEQ ID NO: 13, SEQ ID NO: 14, SEQ ID NO: 15, and an analog or derivative thereof. According to some embodiments, the analog or derivative has at least 90% identity to the selected sequence. According to some particular embodiments, the peptide or polypeptide comprise at least one sequence selected from the group consisting of SEQ ID NO: 11, SEQ ID NO: 12, SEQ ID NO: 13, and an analog or derivative thereof. According to some embodiments, the analog or derivative has at least 90% identity to the selected sequence. According to some particular embodiments, the peptide or polypeptide comprise at least one sequence selected from the group consisting of SEQ ID NO: 11, SEQ ID NO: 12, SEQ ID NO: 13, and an analog or derivative thereof having at least 95% identity to the selected sequence. According to some embodiments said supplementing of the growth medium is performed prior to, during, or following said adding the cells to the growth medium. According to some embodiments the method for culturing comprises growing the cells in suspension. According to some embodiments the method for culturing comprises growing the cells on a scaffold. According to some embodiments the cells are mammalian cells. According to some embodiments, the cells are bovine cells. According to some embodiments, the cells are embryonic stem cells (ESCs). According to some embodiments, the cells are bovine embryonic stem cells (bESCs). According to some embodiments the cells are non-human animal-derived cells. According to some embodiments the cells are bovine-derived cells. According to some embodiments, culturing the cells is in the process of producing cultured meat.

The present invention provides, according to a further aspect, a fusion polypeptide comprising a sequence selected from the group consisting of SEQ ID NO: 10, SEQ ID NO: 14, SEQ ID NO: 15, and an analog or derivative thereof. According to some embodiments, the analog or derivative has at least 90% identity to the selected sequence. According to some embodiments, the fusion polypeptide is for use in enhancing cell growth in cell culture or tissue culture. According to some embodiments the cell culture comprises suspension culture. According to some particular embodiments, the cell culture is used to produce cultured meat. According to some embodiments the tissue culture comprises cultured meat. According to some embodiments, the fusion polypeptide is for use in replacing all or part of the insulin used as a growth factor in cell culture or tissue culture. According to some particular embodiments, the fusion polypeptide is for use in enhancing cell growth in cell culture, at a low concentration relative to insulin concentration in cell culture. According to some embodiments, the low concentration in cell culture comprises less than 0.5, 0.25, less than a 0.15, or less than 0.1 of the insulin molar concentration in cell culture. According to some embodiments, the fusion polypeptide for replacing insulin is provided at a weight concentration of about 0.1 μg/ml to 1 μg/ml. According to other embodiments, the fusion polypeptide for replacing insulin is provided at a molar concentration of about 5 nM to 200 nM.

The present invention provides, according to a further aspect, a peptide comprising SEQ ID NO: 4, or a conjugate or a fusion thereof. According to some embodiments, the peptide consists of 30-200, 30-100, 30-50 or 30-40 amino acid residues. According to some embodiments, the peptide consists of SEQ ID NO: 4. Each option represents a separate embodiment of the present invention. According to some embodiments, a polynucleotide is provided which encodes for the peptide. According to some embodiments, a polynucleotide comprising SEQ ID NO: 23 is provided, or an analog or derivative having at least 80%, at least 85%, at least 90% or at least 95% identity thereto. Each option represents a separate embodiment of the present invention. According to some embodiments, the peptide is for use as in enhancing cell growth in cell culture or tissue culture. According to some particular embodiments, the cell culture is used to produce cultured meat. According to some embodiments, the tissue culture comprises cultured meat. According to some particular embodiments, the peptide is for use in enhancing cell growth in cell culture, at a low concentration relative to insulin concentration in cell culture. According to some embodiments, the low concentration in cell culture comprises less than 0.5, 0.25, less than a 0.15, or less than 0.1 of the insulin molar concentration in cell culture. According to some embodiments, the peptide comprising SEQ ID NO: 4 is used for enhancing cell growth in cell culture at a weight concentration of about 0.1 μg/ml to 1 μg/ml. According to other embodiments, the peptide comprising SEQ ID NO: 4 is used for enhancing cell growth in cell culture at a molar concentration of about 5 nM to 200 nM.

According to a further aspect of the invention, there is provided a peptide, or a fusion polypeptide thereof, for use in enhancing cell growth in culture, wherein the culture comprises a serum-free cell-growth medium, and wherein the peptide or the fusion polypeptide enhances cell growth at a reduced molar concentration relative to insulin concentration in cell culture. According to some embodiments the cell culture is used in the process of producing cultured meat. According to some embodiments, the low molar concentration in culture is less than 0.33, less than 0.25, less than a 0.15, less than 0.1, or less than 0.05 of the effective insulin molar concentration in culture. According to some embodiments, the serum-free cell-growth medium comprises said peptide or fusion polypeptide at a weight concentration of about 0.1 μg/ml to 1 μg/ml. According to other embodiments, the serum-free cell-growth medium comprises said peptide or fusion polypeptide at a molar concentration of about 5 nM to 200 nM. According to some embodiments, the peptide or fusion polypeptide has a thermal stability equal to or better than the thermal stability of mammalian insulin.

Further embodiments and the full scope of applicability of the present invention will become apparent from the detailed description given hereinafter. However, it should be understood that the detailed description and specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE FIGURES

The novel features described herein are set forth with particularity in the appended claims. A better understanding of the characteristics and advantages of the features described herein will be obtained by reference to the following detailed description that sets forth illustrative examples, in which the principles of the features described herein are utilized, and the accompanying drawings of which:

FIG. 1 is a bar-chart showing the relative growth of bESCs in 2D cell culture following 2 cell passages, for cells grown with media containing recombinant human insulin (“insulin”) or different fusion polypeptides, at a concentration of 2.5 μg/ml (captioned “1×”) or at a 5-fold reduced weight concentration (captioned “0.2×”) as indicated in the chart. The growth level of the cells cultivated in media containing insulin at the 1× concentration was set as the baseline growth level.

FIG. 2 is a bar-chart showing the relative growth of bESCs in a suspension cell culture for 4 days, with media containing insulin or different alternative fusion polypeptides, at a concentration of 2.5 μg/ml (1×) or at a 5-fold reduced weight concentration (0.2×) as indicated in the chart. The growth level of the cells cultivated in media containing human insulin at the 1× concentration was set as the baseline growth level. The mean growth level of two repeats is presented.

FIG. 3 is a bar-chart showing the relative growth of bESCs in suspension culture for 4 days, with media containing WT insulin or alternative fusion polypeptides at a 10-fold reduced concentration (0.1×) relative to the baseline concentration from FIGS. 1 and 2 (2.5 μg/ml, “1×”). The growth level of the cells cultivated in media containing reduced concentration WT insulin was set as the baseline growth level. The mean growth level of two repeats is presented.

### DETAILED DESCRIPTION OF THE INVENTION

In the following description, certain specific details are set forth in order to provide a thorough understanding of various embodiments. However, one skilled in the art will understand that the embodiments provided may be practiced without these details. Unless the context requires otherwise, throughout the specification and claims which follow, the word “comprise” and variations thereof, such as, “comprises” and “comprising” are to be construed in an open, inclusive sense, that is, as “including, but not limited to.” As used in this specification and the appended claims, the singular forms “a,” “an,” and “the” include plural referents unless the content clearly dictates otherwise. It should also be noted that the term “or” is generally employed in its sense including “and/or” unless the content clearly dictates otherwise. Further, headings provided herein are for convenience only and do not interpret the scope or meaning of the claimed embodiments.

As used herein the term “about” refers to an amount that is near the stated amount by 10% or less.

The terms “polypeptide” and “fusion polypeptide”, are used interchangeably to refer to a polymer of amino acid residues. As used herein, the term “peptide” generally refers to an amino acid sequence which is derived from a particular source, or which performs a specific function. Peptides according to the present invention may include new sequences, or peptides which are novel as cell-growth enhancers or insulin alternatives, e.g., SEQ ID NO: 2, SEQ ID NO: 3, SEQ ID NO: 4, and the like. Other peptides referred to herein include linkers, solubility enhancers, permeability enhancers, carrier polypeptides, binding peptides, and the like. As used herein, polypeptide generally refers to an amino acid sequence which includes the at least two fused sequences, e.g., a peptide provided according to the present invention, and at least one other peptide selected from linkers, solubility enhancers, permeability enhancers, and carrier polypeptides. Peptides or polypeptides according to the present invention may include amino acid residues including natural and/or non-natural amino acid residues. The terms also include post-expression modifications of the polypeptide, for example, glycosylation, sialylation, acetylation, phosphorylation, and the like. According to some embodiments, the polypeptides of the invention do not comprise post-expression modifications. According to some embodiments, the polypeptides may contain modifications with respect to a native or natural sequence, as long as the protein maintains the desired activity. These modifications may be deliberate, as through site-directed mutagenesis, or may be accidental, such as through mutations of hosts which produce the proteins or errors due to PCR amplification.

Percent (%) sequence identity with respect to a reference polypeptide sequence is the percentage of amino acid residues in a candidate sequence that are identical with the amino acid residues in the reference polypeptide sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are known, for instance, using publicly available computer software such as BLAST, BLAST-2, ALIGN or Megalign (DNASTAR) software. Appropriate parameters for aligning sequences can be determined, including algorithms needed to achieve maximal alignment over the full length of the sequences being compared.

The terms “homologous”, “homology” or “percent homology” when used herein to describe an amino acid sequence or a nucleic acid sequence, relative to a reference sequence, can be determined using the formula described for example by Karlin and Altschul (Proc. Natl. Acad. Sci. USA 87:2264-2268, 1990, modified as in Proc. Natl. Acad. Sci. USA 90:5873-5877, 1993). Such a formula is incorporated into the basic local alignment search tool (BLAST) programs of Altschul et al. (J. Mol. Biol. 215:403-410, 1990). Percent homology of sequences can be determined using the most recent version of BLAST, for example.

In some embodiments, amino acid sequence variants of the polypeptides provided herein are contemplated. A variant typically differs from a polypeptide specifically disclosed herein in one or more substitutions, deletions, additions and/or insertions. Such variants can be naturally occurring or can be synthetically generated, for example, by modifying one or more of the above polypeptide sequences of the invention and evaluating one or more biological activities of the polypeptide as described herein, and/or using any of a number of known techniques. For example, it may be desirable to improve the binding affinity and/or other biological properties of the peptide or polypeptide. Amino acid sequence variants of a polypeptide may be prepared by introducing appropriate modifications into the nucleotide sequence encoding it, or by peptide synthesis. Such modifications include, for example, deletions from, and/or insertions into and/or substitutions of residues within the amino acid sequences of the peptide or polypeptide. Any combination of deletion, insertion and substitution can be made to arrive at the final construct, provided that the final sequence possesses the desired characteristics, e.g., promoting of cell growth.

The terms “specific binding” or “specifically binds” or “binds”, as used herein, refer to a peptide, polypeptide, or protein, binding to a specific receptor, or another moiety, with greater affinity than to other receptors or moieties. Typically, the peptide “specifically binds” when the equilibrium dissociation constant (KD) for binding is about 1×10⁻⁸ M or less, for example about 1×10⁻⁹ M or less, about 1×10⁻¹⁰ M or less, about 1×10⁻¹¹ M or less, or about 1×10⁻¹² M or less, typically with the KD that is at least one hundred-fold less than its KD for binding to a non-specific receptor. The KD may be measured using standard procedures.

The polypeptides, conjugates and constructs described herein can be encoded by a nucleic acid. A nucleic acid is a type of polynucleotide comprising two or more nucleotide bases. In certain embodiments, the nucleic acid is a component of a vector that can be used to transfer the polypeptide-encoding polynucleotide into a cell. As used herein, the term “vector” refers to a nucleic acid molecule capable of transporting another nucleic acid to which it has been linked. One type of vector is a genomically integrated vector, or “integrated vector,” which can become integrated into the chromosomal DNA of the host cell. Another type of vector is an “episomal” vector, e.g., a nucleic acid capable of extra-chromosomal replication. Vectors capable of directing the expression of genes to which they are operatively linked are referred to herein as “expression vectors.” Suitable vectors comprise plasmids, bacterial artificial chromosomes, yeast artificial chromosomes, viral vectors and the like. In the expression vectors, regulatory elements such as promoters, enhancers, polyadenylation signals for use in controlling transcription can be derived from mammalian, microbial, viral or insect genes. The ability to replicate in a host, usually conferred by an origin of replication, and a selection gene to facilitate recognition of transformants may additionally be incorporated. Vectors derived from viruses, such as lentiviruses, retroviruses, adenoviruses, adeno-associated viruses, and the like, may be employed. Plasmid vectors can be linearized for integration into a chromosomal location. Vectors can comprise sequences that direct site-specific integration into a defined location or restricted set of sites in the genome (e.g., AttP-AttB recombination). Additionally, vectors can comprise sequences derived from transposable elements.

The terms “polynucleotide,” “oligonucleotide,” “nucleic acid” and “nucleic acid molecule” are used interchangeably herein to include a polymeric form of nucleotides, either ribonucleotides or deoxyribonucleotides. These terms refer only to the primary structure of the molecule.

“Vector” refers to a polynucleotide capable of being duplicated within a biological system or that can be moved between such systems. Vector polynucleotides typically contain elements, such as origins of replication, polyadenylation signal or selection markers, that function to facilitate the duplication or maintenance of these polynucleotides in a biological system, such as a cell, virus, animal, plant, and reconstituted biological systems utilizing biological components capable of duplicating a vector. The vector polynucleotide may be DNA or RNA molecules, cDNA, or a hybrid of these, single-stranded or double-stranded.

“Expression vector” refers to a vector that can be utilized in a biological system or in a reconstituted biological system to direct the translation of a polypeptide encoded by a polynucleotide sequence present in the expression vector.

A “host cell,” as used herein, denotes an in vivo or in vitro prokaryotic cell, eukaryotic cell, or a cell from a multicellular organism (e.g., a cell line) cultured as a unicellular entity, which prokaryotic or eukaryotic cells can be, or have been, used as recipients for nucleic acid (e.g., an expression vector that comprises a nucleotide sequence encoding a polypeptide of the present disclosure), and include the progeny of the original cell which has been genetically modified by the nucleic acid. It is understood that the progeny of a single cell may not necessarily be completely identical in morphology or in genomic or total DNA complement as the original parent, due to natural, accidental, or deliberate mutation. A “recombinant host cell” (also referred to as a “genetically modified host cell”) is a host cell into which has been introduced a heterologous nucleic acid, e.g., an expression vector. For example, a genetically modified prokaryotic or eukaryotic host cell is genetically modified by virtue of introduction into a suitable host cell of a heterologous nucleic acid, e.g., an exogenous nucleic acid that is foreign to the host cell, or a recombinant nucleic acid that is not normally found in the host cell.

The terms “clean meat” or “cultured meat” or “in-vitro meat” are interchangeably used herein to describe meat grown from in vitro non-human, animal cell culture, instead of from slaughtered animals. Additional terms that may be used in the art to describe meat grown from in vitro animal cell culture include lab-grown meat, test tube meat, tube steak, synthetic meat, cell-cultured meat, tissue engineered meat, engineered meat, meat analog and manmade meat.

As used herein the term “exogenous” with regard to a supplement refers to a substance/compound which is taken up by a cell from a culture medium. The exogenous supplement can be a compound naturally present within the cell (endogenous compound) or a compound not naturally found in a non-human-animal-derived cell (heterologous compound).

As used herein, the term “natural colorant” refers to a pigment that can be found in natural sources, including plants, algae, fungi and the like. It is to be explicitly understood that the natural colorant of the present invention can be derived from the natural source or can be synthesized chemically.

The terms “meat” when used alone, “meat-like”, and “cultured meat” are used herein interchangeably and refer to meat derived from any non-human-animal, including, for example, bovine, sheep, swine, poultry, shellfish and fish.

The term “cell growth” as used herein, refers to at least one of cell proliferation, cell differentiation, and cell maturation. The terms “enhancing cell growth” and “promoting cell growth” are used herein interchangeably and refer to promotion of any type of cell development or proliferation, including, but not limited to, acceleration of cell cycle, enhancement of cell survival rate, maintaining and/or enhancement of cell viability, enhancement of cell size, enhancement of cell differentiation, enhancement of cell maturation, enhancement of cell proliferation, and any combination thereof. Each possibility represents a separate embodiment.

Analogs and derivatives of the peptides are also within the scope of the present application. These include but are not limited to conservative and non-conservative substitutions of amino acids, modification of the peptide's terminal (e.g. acylation of N-terminus, amidation of C-terminus etc.), insertion and deletion of amino acids within the sequence, cyclization, modification of a peptide bond, and combination of two or more such modification. Such modification and the resultant peptide analog or derivative are within the scope of the present invention as long as they confer, or even improve the activity or stability of the peptide, polypeptide or fusions thereof.

The term “analog” indicates a molecule which has the amino acid sequence according to the invention except for one or more amino acid changes, and that retains the activity of the original molecule, in particular with regard to promotion of cell growth in culture. According to some embodiments, the analog consists of a sequence at least 90% identical to the original sequence. According to some embodiments, the analog has at least 91%, at least 92%, at least 93%, at least 94%, at least 95%, at least 96%, at least 97%, at least 98%, or at least 99% identity to the original sequence. According to some embodiments, the analog comprises modification selected from of: 1-5 deletions of amino acids, 1-5 substitutions of amino acids, 1-5 additions of amino acids, addition of a linker, and combinations thereof. Each possibility represents a separate embodiment of the present invention. The design of appropriate “analogs” may be computer assisted and may involve at least one artificial intelligence (AI) algorithm.

Reference to a particular “analog” indicates a peptide or a polypeptide that has the same, or a similar activity as the original unmodified sequence. “Analogs” of the peptides and polypeptides of the invention also include modified peptides (with amino acid substitutions, both conservative and non-conservative as described below) that have the same or improved activity, at least in one aspect, as a wild-type or unmodified peptide. “Salts” of the peptides of the invention are also included in its scope and are organic and inorganic salts.

Natural coded amino acids and their derivatives are represented by three-letter or one-letter codes according to IUPAC conventions. When there is no indication, the L isomer is used. The D isomers are indicated by “D” before the residue abbreviation.

Conservative substitutions of amino acids as known to those skilled in the art are within the scope of the present invention. Conservative amino acid substitutions include replacement of one amino acid with another having the same type of functional group or side chain, e.g., aliphatic, aromatic, positively charged, negatively charged. These substitutions may enhance oral bioavailability, penetration into the islets, targeting to specific beta cell populations, immunogenicity, and the like. One of skill will recognize that individual substitutions, deletions or additions to a peptide, polypeptide, or protein sequence which alters, adds or deletes a single amino acid or a small percentage of amino acids in the encoded sequence is a “conservatively modified variant” where the alteration results in the substitution of an amino acid with a chemically similar amino acid. Conservative substitution tables providing functionally similar amino acids are well known in the art.

The following six groups each contain amino acids that are conservative substitutions for one another:
1) Alanine (A), Serine(S), Threonine (T);
2) Aspartic acid (D), Glutamic acid (E);
3) Asparagine (N), Glutamine (Q);
4) Arginine (R), Lysine (K);
5) Isoleucine (I), Leucine (L), Methionine (M), Valine (V); and
6) Phenylalanine (F), Tyrosine (Y), Tryptophan (W).

“Derivatives” of the peptides and polypeptides of the invention as used herein covers derivatives which may be prepared from the functional groups which occur as side chains on the residues or the N- or C-terminal groups, by means known in the art, and are included in the invention as long as they do not destroy the activity or stability of the peptide or polypeptide, and do not confer toxic properties on compositions containing it.

These derivatives may include, for example, aliphatic esters of the carboxyl groups, amides of the carboxyl groups produced by reaction with ammonia or with primary or secondary amines, N-acyl derivatives of free amino groups of the amino acid residues formed by reaction with acyl moieties (e.g., alkanoyl or carbocyclic aroyl groups), or O-acyl derivatives of free hydroxyl group (e.g., that of seryl or threonyl residues) formed by reaction with acyl moieties. Other modification may result from production of the peptides, polypeptides and fusions thereof using recombinant techniques.

The peptide of the present invention may be produced by any method known in the art, including recombinant and synthetic methods. Synthetic methods include exclusive solid phase synthesis, partial solid phase synthesis, fragment condensation, or classical solution synthesis, all well known to one skilled in the art. The resulted peptide sequence is confirmed by methods known to one skilled in the art, for example amino acid sequencing.

In some embodiments, recombinant protein techniques are used to generate the peptides, polypeptides and fusion polypeptides of the present invention. Recombinant techniques are described, for example, by Puetz and Wurm, (2019) Processes 7(8), 476; O' Flaherty et al. (2020) Biotechnology Advances, Volume 43; Burnett and Burnett (2020) Plants People Planet 2(2), 121-132; Gnoth et al. (2008) Bioproc, and Biosys. Engin. 31, 21-39; Yang et al. (2021) App. Micro, and Biotech. 105, 6607-6626; and Tripathi and Shrivastava (2019) Front. Bioeng. Biotechnol., Section Synthetic Bio., Volume 7.

According to some embodiments of the invention, there is provided a peptide comprising SEQ ID NO: 2 and an analog or derivative thereof. According to some embodiments, the analog or derivative has at least 90% identity to the selected sequence. According to some embodiments of the invention there is provided a peptide comprising SEQ ID NO: 3 and an analog or derivative thereof. According to some embodiments, the analog or derivative has at least 90% identity to the selected sequence. According to some embodiments of the invention there is provided a peptide comprising SEQ ID NO: 4 and an analog or derivative thereof. According to some embodiments, the analog or derivative has at least 90% identity to the selected sequence.

According to some further embodiments of the present invention, there is provided a peptide, comprising a sequence selected from the group consisting of SEQ ID NO: 2, SEQ ID NO: 3, SEQ ID NO: 4, a combination thereof, and an analog or derivative thereof. According to some embodiments, the analog or derivative has at least 90% identity to the selected sequence.

According to some further embodiments of the invention there is provided a peptide comprising SEQ ID NO: 5 and an analog or derivative thereof. According to some embodiments, the analog or derivative has at least 90% identity to the selected sequence. According to some embodiments of the invention there is provided a peptide comprising SEQ ID NO: 6 and an analog or derivative thereof. According to some embodiments, the analog or derivative has at least 90% identity to the selected sequence. According to some particular embodiments of the invention there is provided a peptide, comprising a sequence selected from the group comprising SEQ ID NO: 2, SEQ ID NO: 3, SEQ ID NO: 4, SEQ ID NO: 5, SEQ ID NO: 6, a combination thereof, and an analog or derivative thereof. According to some embodiments, the analog or derivative has at least 90% identity to the selected sequence.

According to some embodiments of the present invention, the peptide may be fused to or conjugated to at least one of a proteinaceous or non-proteinaceous moiety or macromolecule that is different therefrom in type, source, function, and/or intended use. According to some embodiments, the moiety comprises a small molecule such as a detectable probe or a tag that can be used to identify, monitor and/or select the fusion peptide or the heterologous moiety. Each possibility represents a separate embodiment. According to some embodiments, the macromolecule may include an amino acid sequence, a nucleic acid sequence, at least one lipid, a polysaccharide, or any combination thereof. According to some embodiments, the amino acid sequence, also referred to as “heterologous sequence”, includes amino-terminal, carboxyl-terminal and/or side-chain fusions or conjugates ranging in length from one residue to polypeptides containing a hundred or more residues, as well as intra-sequence insertions of single or multiple amino acid residues.

The term “heterologous” in reference to peptide conjugates and fusion peptides and polypeptides, therefore, means a different moiety that is attached to the peptide or polypeptide. In some embodiments of the present invention the heterologous moiety comprises a stretch of amino acids that is different in type, source, function, and/or intended use from the amino acid sequence of the peptide or polypeptide to which the heterologous moiety is conjugated. According to some embodiments, the heterologous moiety is a heterologous sequence consisting of amino acid residues. According to some embodiments, the heterologous sequence comprises 5-100 amino acid residues. According to some embodiments, the heterologous sequence consists of 5-100 amino acid residues.

According to some embodiments, the heterologous sequence comprises a peptide tag. According to some embodiments, the peptide tag is selected from the list consisting of poly-histidine tag (HIS-tag), histidine-glutamine tag (HQ-tag), histidine-asparagine tag (HN-tag), and HAT tag. According to some particular embodiments, the peptide tag is a HIS-tag, comprising between 2-20, between 2-10, between 4-15, between 4-10, between 4-8, or between 10-20 Histidine amino acid residues. Each possibility represents a separate embodiment.

According to some embodiments, the heterologous moiety or sequence is fused to the peptide or polypeptide through a linker. According to some embodiments, the linker comprises between 2-50, between 2-30, between 2-15, between 2-10, between 5-30, between 5-20, between 5-15, between 5-10, between 10-30, between 10-25, between 10-20, between 15-30, between 15-25, between 15-20, or between 30-50 amino acid residues. Each possibility represents a separate embodiment of the present invention. According to some embodiments, the linker comprises serine and asparagine residues.

According to some embodiments, the linker or the heterologous moiety comprises a cleavable sequence that may be cleavable under certain conditions. According to some embodiments, the cleavable sequence is a protease cleavage site. According to some embodiments, the heterologous sequence comprises a protease cleavage site. According to some embodiments, the protease cleavage site is recognizable by Tobacco Etch Virus (TEV) protease. According to some embodiments, the protease cleavage site consists of SEQ ID NO: 9 or an analog or derivative thereof.

According to some embodiments, the heterologous sequence comprises SEQ ID NO: 8, or an analog or derivative thereof. According to some embodiments, the analog or derivative has at least 90% identity to SEQ ID NO: 8.

According to some embodiment of the present invention, there is provided a fusion polypeptide. The fusion polypeptide comprises a peptide comprising a sequence selected from the group comprising SEQ ID NO: 2, SEQ ID NO: 3, SEQ ID NO: 4, SEQ ID NO: 5, SEQ ID NO: 6, and an analog or derivative thereof. According to some embodiments, the analog or derivative has at least 90% identity to the selected sequence, and further comprises a heterologous amino acid sequence. According to some embodiments, the heterologous sequence includes between 5-200 amino acid residues. According to some embodiments the heterologous sequence comprises between 5-100, between 5-75, between 5-50, between 5-40, between 5-30, between 5-25, between 10-100, between 10-50, between 10-20, between 20-200, between 20-150, between 20-100, between 20-50, between 50-200, between 50-150, between 50-120, between 50-100, between 100-200, or between 100-150 amino acid residues.

According to some embodiments the heterologous sequence comprises at least one moiety selected from a solubility-enhancing peptide, a carrier polypeptide, and a permeability enhancing moiety. As used herein, the term “solubility-enhancing peptide” refers to an amino-acid chain residue which is fused to a peptide of interest (referred to herein also as “growth-enhancing peptide”), such as the growth-enhancing peptides of the present invention, which assists in correct folding of the peptide of interest and inhibits or reduces the formation of proteinaceous aggregates within a liquid environment, such as inclusion bodies and the like. Solubility-enhancing peptides may include at least one of maltose-binding protein (MBP), oleosin, low-molecular-weight protamine, HaloTag, glutathione S-transferase, protein disulfide isomerase (PDI), b′a′domain of PDI (PDIb′a′), N-utilization substance protein A, ELK16, thioredoxin, Fh8 protein (SEQ ID NO: 7), a combination of one or more thereof. In some particular embodiments, the solubility-enhancing peptide comprises SEQ ID NO: 7, or an analog or derivative thereof. According to some embodiments, the analog or derivative has having at least 90% identity to SEQ ID NO: 7.

As used herein, the term “carrier polypeptide” refers to an amino acid sequence that is fused to, or conjugated to, a peptide or polypeptide of interest and alters or improves at least one of its properties, for example solubility, stability or permeability.

“Permeability” refers to the ability of an agent or substance to penetrate, pervade, or diffuse through a barrier, membrane, or a skin layer. A “cell permeability moiety”, a “permeability enhancing moiety” or a “cell-penetration moiety” refers to any molecule known in the art which is able to facilitate or enhance penetration of molecules through membranes. Non-limitative examples include: hydrophobic moieties such as lipids, fatty acids, steroids and bulky aromatic or aliphatic compounds; hydrophilic moieties such as Arginine residues or guanidino-containing moieties; moieties which may have cell-membrane receptors or carriers, such as steroids, vitamins and sugars; natural and non-natural amino acids and transporter peptides.

The permeability-enhancing moiety may be connected to any location of the peptide sequence. According to some specific embodiments, the permeability-enhancing moiety is connected to the N-terminus or C-terminus of the peptide or polypeptide. According to other embodiments, the permeability-enhancing moiety is connected to a side chain of an amino acid residue of the peptide or polypeptide. Each possibility represents a separate embodiment of the present invention.

For the intended applications, the peptides and fusion polypeptides of the present invention should be developable as biological substances. This includes that they are stable enough to survive a lengthy, large-scale production process as well as transportation and storage without aggregation or loss of activity.

According to some particular embodiments of the invention, the fusion polypeptide comprises an amino acid sequence selected from SEQ ID NO: 11, SEQ ID NO: 12, SEQ ID NO: 13, SEQ ID NO: 14, SEQ ID NO: 15, and an analog or derivative thereof. According to some embodiments, the analog or derivative has at least 90% identity to the selected sequence. According to further particular embodiments, the fusion polypeptide comprises an amino acid sequence selected from SEQ ID NO: 11, SEQ ID NO: 12, SEQ ID NO: 13, and an analog or derivative thereof. According to some embodiments, the analog or derivative has at least 90% identity to the selected sequence.

According to some embodiments of the present invention, a polynucleotide is provided, encoding for at least one growth-enhancing peptide or polypeptide as described above. According to some particular embodiments the polynucleotide encodes for at least one of SEQ ID NO: 2, SEQ ID NO: 3, SEQ ID NO: 4, SEQ ID NO: 5, SEQ ID NO: 6, SEQ ID NO: 11, SEQ ID NO: 12, SEQ ID NO: 13, SEQ ID NO: 14, SEQ ID NO: 15, and an analog or derivative thereof. According to some embodiments, the analog or derivative has at least 90% identity to the selected amino acid sequence. According to further particular embodiments the nucleic acid sequence encodes for at least one fusion polypeptide comprising SEQ ID NO: 2, SEQ ID NO: 3, or SEQ ID NO: 4, and a heterologous amino acid sequence fused thereto. According to some embodiments the nucleic acid sequence encodes for at least one fusion polypeptide selected from SEQ ID NO: 11, SEQ ID NO: 12, or SEQ ID NO: 13. According to some particular embodiment the polynucleotide is selected from SEQ ID NO: 21, SEQ ID NO: 22, SEQ ID NO: 23, and an analog or derivative thereof. According to some embodiments, the analog or derivative has at least 80% identity to the selected nucleic acid sequence.

According to some embodiments, at least one polynucleotide described above is included within a recombinant vector. According to some embodiments, the recombinant vector is an expression vector. According to some further embodiments, the at least one polynucleotide or recombinant vector are transfected or transformed into a host cell. According to some embodiments, the host cell is a prokaryotic cell. According to further particular embodiments, the host cell is an E. coli bacteria.

According to some embodiments of the present invention, the peptide or polypeptide comprising a sequence selected from SEQ ID NO: 2, SEQ ID NO: 3, SEQ ID NO: 4, SEQ ID NO: 5, SEQ ID NO: 6, SEQ ID NO: 11, SEQ ID NO: 12, SEQ ID NO: 13, SEQ ID NO: 14, SEQ ID NO: 15, an analog or derivative thereof, and a combination thereof, is used as a factor for promoting cell growth. According to some embodiments, the analog or derivative has at least 90% identity to the selected sequence. According to some embodiments of the present invention, the peptide comprising a sequence selected from SEQ ID NO: 2, SEQ ID NO: 3, SEQ ID NO: 4, SEQ ID NO: 5, SEQ ID NO: 6, an analog or derivative thereof, and a combination thereof, is used as a factor for promoting cell growth. According to some particular embodiments of the present invention, the peptide comprising a sequence selected from SEQ ID NO: 2, SEQ ID NO: 3, SEQ ID NO: 4, and an analog or derivative thereof, is used as a factor for promoting cell growth. According to some embodiments of the present invention, the polypeptide comprising a sequence selected from SEQ ID NO: 11, SEQ ID NO: 12, SEQ ID NO: 13, SEQ ID NO: 14, SEQ ID NO: 15, an analog or derivative thereof, and a combination thereof, is used as a factor for promoting cell growth. According to some particular embodiments of the present invention, the polypeptide comprising a sequence selected from SEQ ID NO: 11, SEQ ID NO: 12, SEQ ID NO: 13, and an analog or derivative thereof, is used as a factor for promoting cell growth.

According to some embodiments, a combination of one or more peptides or polypeptides are used for enhancing cell growth, selected from the peptides or polypeptides comprising sequences SEQ ID NO: 2, SEQ ID NO: 3, SEQ ID NO: 4, SEQ ID NO: 11, SEQ ID NO: 12, SEQ ID NO: 13, and an analog or derivative thereof. According to some embodiments, the analog or derivative has at least 90% identity to the selected sequence. According to some exemplary embodiments, SEQ ID NO: 2 and SEQ ID NO: 3 are used conjointly for enhancing cell growth. According to further exemplary embodiments, SEQ ID NO: 2 and SEQ ID NO: 4, SEQ ID NO: 3 and SEQ ID NO: 4, SEQ ID NO: 2 and SEQ ID NO: 11, SEQ ID NO: 3 and SEQ ID NO: 12; SEQ ID NO: 4 and SEQ ID NO: 13, SEQ ID NO: 2 and SEQ ID NO: 12, SEQ ID NO: 2 and SEQ ID NO: 13, SEQ ID NO: 3 and SEQ ID NO: 11, SEQ ID NO: 3 and SEQ ID NO: 13, SEQ ID NO: 4 and SEQ ID NO: 11, or SEQ ID NO: 4 and SEQ ID NO: 12, are used for enhancing cell growth. Each possibility represents a separate embodiment of the invention. Any combination of more than two of the peptides or polypeptides may be used to enhance cell growth. According to some embodiments, the combination of peptides or polypeptides that is used for enhancing cell growth comprises a single relatively elongated polypeptide that contains the two or more combined peptides or polypeptides. According to some embodiments, the combination of peptides or polypeptides that is used for enhancing cell growth comprises separate peptides or polypeptides for each of the two or more combined peptides or polypeptides.

According to some particular embodiments, the peptide or polypeptide is for use in enhancing cell growth in a cell culture or a tissue culture. According to some embodiments, the cell culture is used in the process of producing cultured meat. According to some further embodiments, the tissue culture comprises cultured meat. According to some embodiments of the invention, the peptide or polypeptide is used to promote cell growth in cell culture or tissue culture at a low concentration relative to insulin concentration used as a growth-enhancing factor in cell culture. Insulin is typically used in cell culture, or in cell-culture medium, at a concentration in the range of 2 μg/ml to 30 μg/ml, equivalent to a molar concentration in the range of about 0.3 μM to 6 μM, particularly in the range of 5 μg/ml to 20 μg/ml, equivalent to a molar concentration in the range of about 0.75 μM to 3.5 μM. According to some embodiments, the cell-culture comprises a weight concentration of about 0.1 μg/ml to 1 μg/ml of a polypeptide selected from the group consisting of: SEQ ID NO: 11, SEQ ID NO: 12, and SEQ ID NO: 13. According to some embodiments, the cell-culture comprises a concentration of about 0.25 μg/ml to 0.5 μg/ml of a polypeptide selected from the group consisting of: SEQ ID NO: 11, SEQ ID NO: 12, and SEQ ID NO: 13. According to some embodiments, the cell-culture comprises a concentration of about 5 nM to 200 nM of a polypeptide selected from the group consisting of: SEQ ID NO: 11, SEQ ID NO: 12, and SEQ ID NO: 13. According to some embodiments, the cell-culture comprises a concentration of about 15 nM to 150 nM of a polypeptide selected from the group consisting of: SEQ ID NO: 11, SEQ ID NO: 12, and SEQ ID NO: 13. According to some particular embodiments, the cell-culture comprises a concentration of about 15 nM to 50 nM of a polypeptide selected from the group consisting of: SEQ ID NO: 11, SEQ ID NO: 12, and SEQ ID NO: 13, including each value within the specified range.

According to some embodiments, the cell-culture comprises a weight concentration of about 0.1 μg/ml to 1 μg/ml of a polypeptide selected from the group consisting of: SEQ ID NO: 11, SEQ ID NO: 12, SEQ ID NO: 13, SEQ ID NO: 14, and SEQ ID NO: 15. According to some embodiments, the cell-culture comprises a concentration of about 0.25 μg/ml to 0.5 μg/ml of a polypeptide selected from the group consisting of: SEQ ID NO: 11, SEQ ID NO: 12, SEQ ID NO: 13, SEQ ID NO: 14, and SEQ ID NO: 15. According to some embodiments, the cell-culture comprises a concentration of about 5 nM to 200 nM of a polypeptide selected from the group consisting of: SEQ ID NO: 11, SEQ ID NO: 12, SEQ ID NO: 13, SEQ ID NO: 14, and SEQ ID NO: 15. According to some embodiments, the cell-culture comprises a concentration of about 15 nM to 150 nM of a polypeptide selected from the group consisting of: SEQ ID NO: 11, SEQ ID NO: 12, SEQ ID NO: 13, SEQ ID NO: 14, and SEQ ID NO: 15. According to some particular embodiments, the cell-culture comprises a concentration of about 15 nM to 50 nM of a polypeptide selected from the group consisting of: SEQ ID NO: 11, SEQ ID NO: 12, SEQ ID NO: 13, SEQ ID NO: 14, and SEQ ID NO: 15, including each value within the specified range.

According to some embodiments, the cell-culture, or cell-culture medium, comprises a concentration of about 0.1 μg/ml to 1 μg/ml of a peptide selected from the group consisting of: SEQ ID NO: 2, SEQ ID NO: 3, and SEQ ID NO: 4. According to some embodiments, the cell-culture comprises a concentration of about 0.25 μg/ml to 0.5 μg/ml of a peptide selected from the group consisting of: SEQ ID NO: 2, SEQ ID NO: 3, and SEQ ID NO: 4. According to some embodiments, the cell-culture comprises a concentration of about 5 nM to 200 nM of a polypeptide selected from the group consisting of: SEQ ID NO: 2, SEQ ID NO: 3, and SEQ ID NO: 4. According to some embodiments, the cell-culture comprises a concentration of about 10 nM to 100 nM of a polypeptide selected from the group consisting of: SEQ ID NO: 2, SEQ ID NO: 3, and SEQ ID NO: 4. According to some embodiments, the cell-culture comprises a concentration of about 15 nM to 150 nM of a polypeptide selected from the group consisting of: SEQ ID NO: 2, SEQ ID NO: 3, and SEQ ID NO: 4. According to some particular embodiments, the cell-culture comprises a concentration of about 15 nM to 50 nM of a polypeptide selected from the group consisting of: SEQ ID NO: 2, SEQ ID NO: 3, and SEQ ID NO: 4, including each value within the specified range.

According to some embodiments, the cell-culture comprises a concentration of about 0.1 μg/ml to 1 μg/ml of a peptide selected from the group consisting of: SEQ ID NO: 2, SEQ ID NO: 3, SEQ ID NO: 4, SEQ ID NO: 5, and SEQ ID NO: 6. According to some embodiments, the cell-culture comprises a concentration of about 0.25 μg/ml to 0.5 μg/ml of a peptide selected from the group consisting of: SEQ ID NO: 2, SEQ ID NO: 3, SEQ ID NO: 4, SEQ ID NO: 5, and SEQ ID NO: 6. According to some embodiments, the cell-culture comprises a concentration of about 5 nM to 200 nM of a polypeptide selected from the group consisting of: SEQ ID NO: 2, SEQ ID NO: 3, SEQ ID NO: 4, SEQ ID NO: 5, and SEQ ID NO: 6. According to some embodiments, the cell-culture comprises a concentration of about 10 nM to 100 nM of a polypeptide selected from the group consisting of: SEQ ID NO: 2, SEQ ID NO: 3, SEQ ID NO: 4, SEQ ID NO: 5, and SEQ ID NO: 6. According to some embodiments, the cell-culture comprises a concentration of about 15 nM to 150 nM of a polypeptide selected from the group consisting of: SEQ ID NO: 2, SEQ ID NO: 3, SEQ ID NO: 4, SEQ ID NO: 5, and SEQ ID NO: 6. According to some particular embodiments, the cell-culture comprises a concentration of about 15 nM to 50 nM of a polypeptide selected from the group consisting of: SEQ ID NO: 2, SEQ ID NO: 3, SEQ ID NO: 4, SEQ ID NO: 5, and SEQ ID NO: 6, including each value within the specified range.

According to some embodiments, the low concentration of the peptides or polypeptides in cell culture is a low molar concentration. According to some embodiments, the low molar concentration in cell culture is less than about 0.33, less than about 0.25, less than a about 0.15, less than about 0.1, or less than about 0.05 of the insulin molar concentration used in cell culture. According to some embodiments, the low concentration of the peptide or polypeptide in cell culture is a low weight concentration. According to some embodiments, the low weight concentration is less than about 0.25, less than about 0.15, less than about 0.1, or less than about 0.05 of the insulin weight concentration used in cell culture. According to some embodiments, the peptide or polypeptide according to the present invention is used as a replacement of insulin as a growth-enhancing factor in cell culture.

According to some embodiments, the peptide or polypeptide is used alongside insulin as a growth-enhancing factor in cell culture. According to some particular embodiments, the insulin is added to the cell culture at a concentration in the range of 1 μg/ml to 20 μg/ml, 1 μg/ml to 10 μg/ml, 1 μg/ml to 5 μg/ml, or 5 μg/ml to 10 μg/ml. Each possibility represents a separate embodiment of the invention. According to some embodiments, the peptide or polypeptide is used together with insulin as a growth-enhancing factor, wherein the molar ratio in the cell culture of the peptide or polypeptide to insulin, is about 1:1. According to some further embodiments, the molar ratio in the cell culture of the peptide or polypeptide to insulin, is about 1:2, 1:3, 1:4, 1:5, 2:1, 3:1, 4:1, 5:1, 6:1, 7:1, 8:1, 9:1, 10:1, 12:1, 15:1, 20:1, or more. Each possibility represents a separate embodiment of the invention.

It should be noted that the growth-promoting peptides and fusion polypeptides disclosed above may be used not only for culturing of cells but also in other biological and biomedical processes. These processes include but are not limited to drug development, therapeutic treatment, production of biomaterials, food production processes, development and production of cosmetics, and cosmetic treatment. Each possibility represents a separate embodiment of the present invention. According to some embodiments, the therapeutic treatment comprises wound healing, bone healing, cartilage healing, and the like. According to some embodiments, the cosmetic treatment comprises hair growth, nail growth, and the like.

According to some embodiments, the peptide or polypeptide is for use in promoting cell growth in at least one of wound healing, hair growth, and the like.

According to some embodiments the peptides and fusion polypeptides, or cell culture comprising them, is used in the process of at least one of development of viral vaccines, developing cells for transplantation, production of enzymes, production of hormones, cellular agriculture, molecular biology research, and the like. according to some embodiments, the cell culture comprises mammalian cell culture. According to some particular embodiments, the peptides and fusion polypeptides and cell culture comprising them is used in cellular agriculture selected from cultured milk, cultured meat, and the like. According to further particular embodiments the cell agriculture comprises cultured milk or cultured meat. In some embodiments the cell agriculture comprises cultured meat.

According to some embodiments, the cell culture comprises suspension culture or adherent culture. According to some particular embodiments, the cell culture comprises suspension culture.

According to certain embodiments, the cell culture of any one of the above-described embodiments is grown on a scaffold to form cultured meat. According to some embodiments, the scaffold may be an edible scaffold. Any edible scaffold known in the art for use in cultured meat may be used with the cell culture of the present invention, including edible protein scaffold, edible hydrogel scaffold, edible polysaccharide scaffold and the like. According to some embodiments, the scaffold is derived from plants, algae or microorganisms. Each possibility represents a separate embodiment of the present invention.

According to some embodiments the cells grown in the culture (herein “cultured cells”) comprise non-human-animal-derived cells. The term “non-human animal-derived cells”, refers to cells derived from any non-human-animal, including, for example, bovine, sheep, swine, poultry, shellfish and fish. Other animals which may be a source for cells which may be used in cell culture are also contemplated, particularly, but not limited to, cell culture which is used in the process of producing cultured meat. According to some embodiments, the non-human-animal-derived cells are pluripotent stem cells (PSCs) and/or cells differentiated therefrom. According to further embodiments, the PSCs are embryonic stem cells. According to further embodiments, the PSCs are non-embryonic stem cells (ESCs). According to further embodiments, the PSCs are induced PSCs (iPSCs) reprogrammed from somatic non-human animal cells and/or cells differentiated therefrom.

According to some embodiments, the non-human-derived animal cells comprise mammalian embryonic stem cells. According to some specific embodiments the mammalian embryonic stem cells are bovine Embryonic Stem Cells (bESC).

According to some embodiments, the non-human-animal-derived cells are pluripotent stem cells differentiated to muscle cells. According to certain embodiments, the non-human-animal-derived cells are pluripotent stem cells differentiated to fat cells (adipocytes) and/or its progenitors. According to some embodiments, the non-human animal-derived cells are pluripotent stem cells differentiated to stromal cells (connective tissue) and/or its progenitors. According to certain embodiments, the non-human animal-derived cells are pluripotent stem cells differentiated to endothelial cells (blood vessels) and/or its progenitors. According to some embodiments, the non-human-animal-derived cells are satellite cells differentiated to muscle cells and/or its progenitors. According to certain embodiments, the non-human-animal derived cells are selected from the group consisting of muscle cells, fat cells, stromal cells, fibroblasts, pericytes, endothelial cells, the progenitors of the above cells, and/or a combination thereof.

According to some embodiments of the present invention, a composition including at least one of the growth-enhancing peptides or polypeptides described above, and at least one excipient, buffer or salt is provided. According to some embodiments, the composition is a liquid composition, a semi liquid composition or a dry composition. For example, the composition may include a cream, an aqueous solution, a non-aqueous solution, a gel, a paste, an ointment, a liquid buffer, a powder, a tablet, a capsule, and the like. Each possibility represents a separate embodiment.

According to some embodiments, the composition comprises a medium for growing cells in culture (herein referred to as “cell-culture medium”). According to some embodiments of the invention, there is provided a cell-culture medium including a growth-enhancing peptide comprising an amino acid sequence selected from SEQ ID NO: 2, SEQ ID NO: 3, SEQ ID NO: 4, SEQ ID NO: 5, SEQ ID NO: 6, a combination thereof, and an analog or derivative thereof. According to some embodiments, the analog or derivative has at least 90% identity to the selected sequence. According to some particular embodiments, the cell-culture medium includes a growth-enhancing peptide comprising an amino acid sequence selected from SEQ ID NO: 2, SEQ ID NO: 3, SEQ ID NO: 4, a combination thereof, and an analog or derivative thereof. According to some embodiments, the analog or derivative has at least 90% identity to the selected sequence. According to some embodiments the medium comprises a growth-enhancing fusion polypeptide comprising at least one amino acid sequence selected from SEQ ID NO: 2, SEQ ID NO: 3, SEQ ID NO: 4, and an analog or derivative thereof. According to some embodiments, the analog or derivative has at least 90% identity to the selected sequence, and a heterologous amino acid sequence according to the present invention as described above. According to some particular embodiments of the invention, the cell-culture medium includes a growth-enhancing polypeptide comprising an amino acid sequence selected from SEQ ID NO: 10, SEQ ID NO: 11, SEQ ID NO: 12, SEQ ID NO: 13, SEQ ID NO: 14, SEQ ID NO: 15, a combination thereof, and an analog or derivative thereof. According to some embodiments, the analog or derivative has at least 90% identity to the selected sequence. According to some further embodiments, the cell-culture medium includes a growth-enhancing polypeptide comprising an amino acid sequence selected from SEQ ID NO: 11, SEQ ID NO: 12, SEQ ID NO: 13, a combination thereof, and an analog or derivative thereof. According to some embodiments, the analog or derivative has at least 90% identity to the selected sequence.

According to some embodiments of the invention, the growth-enhancing peptide or polypeptide is present in the cell-culture medium at a low concentration relative to insulin concentration used in mammalian cell culture medium, as defined above. According to some embodiments, the low concentration of the growth-enhancing peptide or polypeptide in the cell-culture medium is a low molar concentration. According to some embodiments, the low molar concentration is less than about 0.33, less than about 0.25, less than a about 0.15, less than about 0.1, or less than 0.05 of the insulin molar concentration used in cell culture medium. According to some embodiments, the low concentration of the growth-enhancing peptide or polypeptide in the cell-culture medium is a low weight concentration. According to some embodiments, the low weight concentration is less than about 0.25, less than about 0.15, less than about 0.1, or less than about 0.05 of the insulin concentration in cell culture.

According to some embodiments, the cell-culture medium includes the growth-enhancing peptide or polypeptide as a replacement of insulin as a growth-enhancing factor i.e., the medium is free of insulin, at least as a growth enhancing factor. According to some alternative embodiments, the cell-culture medium includes both insulin and the growth-enhancing peptide or polypeptide as growth-enhancing factors. According to some particular embodiments, the molar ratio of the growth enhancing peptide or polypeptide to insulin in the cell-culture medium is about 1:1. According to some further embodiments, the molar ratio in the cell-culture medium of the growth-enhancing peptide or polypeptide to insulin, is about 1:2, 1:3, 1:4, 1:5, 2:1, 3:1, 4:1, 5:1, 6:1, 7:1, 8:1, 9:1, 10:1, 12:1, 15:1, 20:1, or more. Each possibility represents a separate embodiment of the invention.

According to some embodiments, the cell-culture medium comprises a carbon source (e.g., glucose and/or glutamine), a nitrogen source (e.g., amino acids), salts (e.g., potassium, sodium and calcium), and vitamins (e.g., riboflavin, thiamine and biotin). According to some particular embodiments, the cell-culture medium comprises at least one supplement selected from iron or a salt thereof, folate, zinc or a salt thereof, selenium or a salt thereof, vitamin D, vitamin E, Coenzyme Q10, a fatty acid, a natural colorant, and cobalamin (vitamin B12). According to some embodiments of the present inventio the cell-culture medium contains less than 5% of animal serum (e.g., fetal bovine serum, horse serum, and the like). According to some embodiments the cell-culture medium contains less than about 4%, less than about 3%, less than about 2%, less than about 1%, less than about 0.5%, or less than about 0.1% of animal serum. According to some particular embodiments, the cell-growth medium is serum-free.

According to further embodiments of the present invention, a method for culturing cells is provided, comprising supplementing a cell growth medium with at least one peptide or fusion polypeptide comprising a sequence selected from SEQ ID NO: 2, SEQ ID NO: 3, SEQ ID NO: 4, SEQ ID NO: 5, SEQ ID NO: 6, and an analog or derivative thereof, and further comprising adding cells to the growth medium. According to some embodiments, the analog or derivative has at least 90% identity to the selected sequence. According to some particular embodiments, the method comprises supplementing a cell growth medium with at least one peptide or fusion polypeptide comprising a sequence selected from SEQ ID NO: 2, SEQ ID NO: 3, SEQ ID NO: 4, SEQ ID NO: 5, SEQ ID NO: 6, SEQ ID NO: 11, SEQ ID NO: 12, SEQ ID NO: 13, SEQ ID NO: 14, SEQ ID NO: 15, and an analog or derivative thereof. According to some embodiments, the analog or derivative has at least 90% identity to the selected sequence polypeptide. According to some particular embodiments, the method comprises supplementing a cell growth medium with at least one peptide or fusion polypeptide comprising a sequence selected from SEQ ID NO: 2, SEQ ID NO: 3, SEQ ID NO: 4, SEQ ID NO: 11, SEQ ID NO: 12, SEQ ID NO: 13, and an analog or derivative thereof. According to some embodiments, the analog or derivative has at least 90% identity to the selected sequence polypeptide.

According to some embodiments, the cell growth medium is supplemented with the peptide or polypeptide at a low concentration relative to the concentration of insulin in cell growth medium. According to some embodiments the low concentration is a low molar concentration. According to some embodiments the concentration is a low weight concentration.

According to some embodiments, supplementing of the growth medium is performed prior to adding of the cells thereto. According to further embodiments, supplementing of the growth medium is performed concurrently with adding of the cells thereto. According to some embodiments, supplementing of the growth medium is performed following adding of the cells thereto. According to some embodiments supplementing of the growth medium is performed prior to, during, and/or following adding of the cells thereto.

According to some embodiments, adding the cells to the growth medium comprises growing the cells in suspension culture. According to some embodiments, adding the cells to the growth medium comprises growing the cells on a scaffold.

According to some embodiments, the cells comprise mammalian cells, as described herein above. According to some embodiments the cells comprise non-human animal-derived cells, as described herein above. According to some embodiments, the cells are grown in the process of producing cultured meat.

According to some embodiments of the invention, there is provided a method for producing at least one growth-enhancing peptide or polypeptide, the method comprising the steps of transforming a vector comprising a polynucleotide which encodes the growth-enhancing peptide or polypeptide into a bacterial host, growing the bacteria, harvesting the bacteria, and extracting the growth enhancing peptide or polypeptide produced by the bacterial host. According to some particular embodiments, the method comprises transforming a vector comprising a polynucleotide which encodes the growth-enhancing peptide or polypeptide under an inducible promoter into a bacterial host; growing the bacteria until a desired density (for example until an optical density in the range of 0.2-0.8_{OD600} is achieved); activating the inducible promoter for 1-10 hours to produce the growth-enhancing peptide or polypeptide in a temperature in the range of 25° C.-35° C.; and harvesting the bacterial population and extracting the growth-enhancing peptide or polypeptide. According to some embodiments, the produced growth-enhancing peptide or polypeptide comprises at least one sequence selected from SEQ ID NO: 2, SEQ ID NO: 3, SEQ ID NO: 4, SEQ ID NO: 5, SEQ ID NO: 6, SEQ ID NO: 11, SEQ ID NO: 12, SEQ ID NO: 13, SEQ ID NO: 14, SEQ ID NO: 15, and an analog or derivative thereof. According to some embodiments, the analog or derivative has at least 90% identity to the selected sequence polypeptide. According to some particular embodiments, the produced growth-enhancing peptide or polypeptide comprises at least one sequence selected from SEQ ID NO: 2, SEQ ID NO: 3, SEQ ID NO: 4, SEQ ID NO: 11, SEQ ID NO: 12, SEQ ID NO: 13, and an analog or derivative thereof. According to some embodiments, the analog or derivative has at least 90% identity to the selected sequence polypeptide. According to some embodiments, the bacterial host comprises E. coli. According to some embodiments, growing the bacteria prior to induction of the inducible promoter is until an optical density in the range of 0.3-0.7_{OD600}, 0.4-0.6_{OD600}, or 0.45-0.55_{OD600}, is achieved. Each possibility represents a separate embodiment of the invention. According to some embodiments, the inducible promoter comprises an IPTG (Isopropyl ß-D-1-thiogalactopyranoside) promoter. According to some particular embodiments, activating the inducible promoter comprises adding IPTG at a concentration in the range of 0.05-1 mM. in some particular embodiments, the IPTG is added at a concentration of 0.2 mM. According to some embodiments, the induction (i.e., promoter activation) is for 2-8 hours, for 2-6 hours, for 3-5 hours, or for 4 hours. Each possibility represents a separate embodiment of the invention. According to some embodiments, the temperature of the bacterial population during the induction is in the range of 28-32° C.

According to some embodiments, the produced growth-enhancing peptide or polypeptide may comprise a heterologous amino acid sequence fused to an insulin-like peptide via a protease cleavage site, and the method for production may further include cleaving the heterologous amino acid sequence from the insulin-like peptide using a protease. According to some embodiments, the protease cleavage site is a TEV cleavage site. According to some particular embodiments, the cleavage site includes an amino acid sequence comprising SEQ ID NO: 9.

Selected peptides disclosed in the present invention are provided in Table 1 and in the sequence listing.

TABLE 1
selected peptides

| Seq ID #. | Seq | Name |
|---|---|---|
| 1 | VNQHLCGSDLVEALYLVCGERGFFYTPETEEGPRRGIVEQCC | Single-Chain Insulin |
| | HSICSLEQLENYCN | (Glidden et. al, 2018, |
| | | ibid) |
| 2 | PLCGSELVDALELVCHDRGGFYSPTIKEVHTNRKTQSGIVEKC | Peptide 34 |
| | CRKEGCDYAYLTQYCAH | (Uniprot A0A6B9XK06) |
| 3 | QLQVCGGELIDALTEHCGDRGVYTPPRRGRRTRSVGLADACC | Peptide 48 |
| | KNECDENELDRYCNPK | (Uniprot Q5YFK3) |
| 4 | SLEEEWAQIECEVYGRGCPKPDFYDWFERQL | Peptide 83, analog of |
| | | S597 of PDB 8DTL |
| 5 | GSDLVEALYLVLGERGFFYTDPTGGGPRRGIVEQLLHSILSLY | Peptide 41 |
| | QLENYLN | |
| 6 | FVNQHLCGSDLVEALYLVCGERGFFYTDPTGGQPRRGIVEQC | Peptide 76 |
| | CHSICSLYQLENYCN | |
| 7 | MPSVQEVEKLLHVLDRNGDGKVSAEELKAFADDSKCPLDSN | Solubility peptide |
| | KIKAFIKEHDKNKDGKLDLKELVSIL | |
| 8 | HHHHHHMPSVQEVEKLLHVLDRNGDGKVSAEELKAFADDS | Fusion peptide (FP) |
| | KCPLDSNKIKAFIKEHDKNKDGKLDLKELVSILSSSSSSSNNNN | |
| | NNNNNNENLYFQG | |
| 9 | ENLYFQG | TEV protease cleavage |
| | | site |
| 10 | MHHHHHHMPSVQEVEKLLHVLDRNGDGKVSAEELKAFADD | Single-Chain Insulin + |
| | SKCPLDSNKIKAFIKEHDKNKDGKLDLKELVSILSSSSSSSNNN | FP |
| | NNNNNNNENLYFQGFVNQHLCGSDLVEALYLVCGERGFFYT | |
| | PETEEGPRRGIVEQCCHSICSLEQLENYCN | |
| 11 | MGHHHHHHMPSVQEVEKLLHVLDRNGDGKVSAEELKAFAD | 34 + FP |
| | DSKCPLDSNKIKAFIKEHDKNKDGKLDLKELVSILSSSSSSSNN | |
| | NNNNNNNNENLYFQGMPLCGSELVDALELVCHDRGGFYSPTI | |
| | KEVHTNRKTQSGIVEKCCRKEGCDYAYLTQYCAH | |
| 12 | MGHHHHHHMPSVQEVEKLLHVLDRNGDGKVSAEELKAFAD | 48 + FP |
| | DSKCPLDSNKIKAFIKEHDKNKDGKLDLKELVSILSSSSSSSNN | |
| | NNNNNNNNENLYFQGMQLQVCGGELIDALTEHCGDRGVYTP | |
| | PRRGRRTRSVGLADACCKNECDENELDRYCNPK | |
| 13 | MGHHHHHHMPSVQEVEKLLHVLDRNGDGKVSAEELKAFAD | 83 + FP |
| | DSKCPLDSNKIKAFIKEHDKNKDGKLDLKELVSILSSSSSSSNN | |
| | NNNNNNNNENLYFQGMSLEEEWAQIECEVYGRGCPKPDFYD | |
| | WFERQL | |
| 14 | MGHHHHHHMPSVQEVEKLLHVLDRNGDGKVSAEELKAFAD | 41 + FP |
| | DSKCPLDSNKIKAFIKEHDKNKDGKLDLKELVSILSSSSSSSNN | |
| | NNNNNNNNENLYFQGMGSDLVEALYLVLGERGFFYTDPTGG | |
| | GPRRGIVEQLLHSILSLYQLENYLN | |
| 15 | MGHHHHHHMPSVQEVEKLLHVLDRNGDGKVSAEELKAFAD | 76+ FP |
| | DSKCPLDSNKIKAFIKEHDKNKDGKLDLKELVSILSSSSSSSNN | |
| | NNNNNNNNENLYFQGMFVNQHLCGSDLVEALYLVCGERGFF | |
| | YTDPTGGQPRRGIVEQCCHSICSLYQLENYCN | |
| 16 | MGHHHHHHMPSVQEVEKLLHVLDRNGDGKVSAEELKAFAD | 2 + FP |
| | DSKCPLDSNKIKAFIKEHDKNKDGKLDLKELVSILSSSSSSSNN | Negative control |
| | NNNNNNNNENLYFQGMHLCGEDLEEALKLVCGERGFYYVD | |
| | PTDGNPENTVVKRCCEDVCSVEQLERFCN | |
| 17 | MGHHHHHHMPSVQEVEKLLHVLDRNGDGKVSAEELKAFAD | 4 + FP |
| | DSKCPLDSNKIKAFIKEHDKNKDGKLDLKELVSILSSSSSSSNN | |
| | NNNNNNNNENLYFQGMQIQVCGGELIETLQRFCGDRGVWFP | |
| | PNNDQDDRDKDLFDACCKNECNENELDRYCNPK | |
| 18 | MGHHHHHHMPSVQEVEKLLHVLDRNGDGKVSAEELKAFAD | 64 + FP |
| | DSKCPLDSNKIKAFIKEHDKNKDGKLDLKELVSILSSSSSSSNN | |
| | NNNNNNNNENLYFQGMFVNQHYCGSDLVEALYLVCGERGFI | |
| | YYDPTNNQPRRSIWEQCCHNICSLEQLENYCN | |
| 19 | MGHHHHHHMPSVQEVEKLLHVLDRNGDGKVSAEELKAFAD | 74 + FP |
| | DSKCPLDSNKIKAFIKEHDKNKDGKLDLKELVSILSSSSSSSNN | |
| | NNNNNNNNENLYFQGMHLCGEDLEDALRLVCGERGFYFVDE | |
| | TDGNPQNTVLRRCCNDVCSVEQLERFCN | |
| 20 | ATGCACCACCACCACCACCACATGCCGAGCGTGCAGGAAG | Nucleotide encoding |
| | TGGAAAAACTGCTGCACGTGCTGGACCGTAACGGTGATGG | SEQ ID NO. 10 |
| | CAAAGTTAGCGCGGAGGAACTGAAAGCGTTCGCGGACGAT | |
| | AGCAAGTGCCCGCTGGATAGCAACAAGATCAAAGCGTTTA | |
| | TTAAGGAACACGACAAGAACAAAGATGGCAAGCTGGACCT | |
| | GAAAGAGCTGGTTAGCATCCTGAGCAGCAGCAGCAGCAGC | |
| | AGCAATAACAATAATAACAACAACAACAACAACGAAAAC | |
| | CTGTACTTCCAGGGTTTTGTTAACCAACATCTGTGCGGCAG | |
| | CGACCTGGTGGAGGCGCTGTACCTGGTTTGCGGTGAACGT | |
| | GGCTTCTTTTATACCCCGGAGACCGAGGAAGGTCCGCGTCG | |
| | TGGCATCGTGGAGCAGTGCTGCCACAGCATTTGCAGCCTG | |
| | GAGCAACTGGAAAACTATTGCAACTAA | |
| 21 | ATGGGACATCATCACCACCATCATATGCCGTCAGTGCAAG | Nucleotide encoding |
| | AAGTGGAAAAACTCTTACATGTATTAGATCGTAATGGTGAT | SEQ ID NO. 11 |
| | GGCAAGGTTTCGGCTGAAGAGCTGAAAGCATTCGCCGACG | |
| | ATAGCAAATGCCCCCTCGATAGTAATAAAATTAAAGCCTTC | |
| | ATAAAAGAGCATGACAAAAATAAAGATGGAAAATTGGAC | |
| | CTGAAGGAACTTGTATCTATATTGTCATCATCATCCTCGAG | |
| | TTCAAATAACAACAATAATAACAATAATAATAACGAAAAC | |
| | CTCTACTTTCAGGGCATGCCGCTGTGCGGGAGCGAGCTTGT | |
| | TGATGCACTGGAACTGGTTTGTCACGATCGCGGAGGCTTTT | |
| | ACTCTCCTACAATCAAGGAGGTTCATACAAACCGTAAAAC | |
| | GCAATCAGGTATCGTGGAGAAATGCTGCCGCAAAGAGGGC | |
| | TGTGATTACGCGTATTTAACTCAGTACTGTGCACACTAA | |
| 22 | ATGGGACACCATCATCACCATCATATGCCGTCAGTTCAAGA | Nucleotide encoding |
| | GGTCGAAAAGTTGTTGCATGTACTTGATCGTAACGGCGAC | SEQ ID NO. 12 |
| | GGAAAAGTGAGCGCCGAGGAGTTAAAAGCATTCGCAGACG | |
| | ATAGTAAATGCCCATTAGATAGCAACAAAATCAAGGCTTT | |
| | CATAAAAGAGCATGATAAGAATAAGGATGGTAAGCTTGAC | |
| | CTTAAAGAGCTGGTGAGTATACTCTCTAGCAGCAGTAGCTC | |
| | TTCCAACAATAACAACAATAACAATAACAATAATGAGAAT | |
| | TTGTACTTCCAGGGTATGCAACTTCAGGTTTGCGGTGGCGA | |
| | ACTCATAGACGCGTTAACAGAACACTGTGGAGATCGGGGT | |
| | GTTTACACTCCACCTCGTAGAGGGCGTAGAACTCGCTCTGT | |
| | TGGCTTAGCGGACGCTTGCTGTAAGAACGAGTGTGACGAA | |
| | AACGAACTCGACAGATACTGTAACCCCAAGTAA | |
| 23 | ATGGGACACCATCATCATCATCACATGCCTAGCGTGCAAG | Nucleotide encoding |
| | AAGTGGAGAAACTGCTGCACGTATTGGATCGAAACGGCGA | SEQ ID NO. 13 |
| | TGGTAAAGTGTCAGCGGAGGAATTGAAAGCATTCGCGGAC | |
| | GACAGTAAGTGTCCCCTGGACAGCAACAAGATCAAAGCTT | |
| | TTATAAAGGAGCATGACAAAAATAAAGATGGAAAACTGGA | |
| | CTTGAAAGAGTTGGTCAGTATTCTGTCTAGTAGTAGCTCAT | |
| | CAAGTAACAATAACAACAATAACAACAATAATAACGAAAA | |
| | CCTGTATTTTCAGGGCATGAGTCTGGAAGAAGAGTGGGCA | |
| | CAGATAGAATGCGAAGTTTATGGACGTGGTTGCCCGAAAC | |
| | CTGATTTTTATGATTGGTTCGAACGACAACTCTAA | |

### EXAMPLES

### Example 1: Selection of Candidate Peptides

In an attempt to enhance cell growth in cell culture, proteinaceous entities which potentially display growth-enhancing activity were identified. 95 peptides were each selected out of one of following three categories: 1) a variant of single chain insulin (SEQ ID NO: 1, herein SC insulin, (“SCI-a” from Glidden et al. 2018 ibid, with an His^{B10} to Asp substitution); 2) a viral protein or portion thereof which has previously been shown to activate insulin receptor (IR); and, 3) a synthetic amino acid sequence which has previously been shown to activate IR. The highest scoring peptides were selected using an artificial intelligence (AI) algorithm analysis, which estimated expected expression titers and thermal stability of a large dataset of peptides (implying prolonged shelf life).

Following selection of the 95 peptides, an initial, small-scale, bacterial-expression assay was performed. The SC insulin was also included in the bacterial-expression assay, amounting to 96 tested peptides overall. In order to enhance expression titer, each of the 96 peptides was fused to the heterologous amino acid sequence of SEQ ID NO: 8, which includes a solubility-enhancing sequence (SEQ ID NO: 7), as well as a his-tag, a serine-asparagine linker, and a protease cleavage site (SEQ ID NO: 9). Vectors containing each of the fusion polypeptides under an IPTG inducible promoter were then respectively transformed into E. coli bacteria. The bacteria were maintained at 30° C. Induction was initiated when bacterial density reached 0.5 OD600, at a concentration of 0.2 mM IPTG. The bacteria were harvested 4 hours following induction, and the polypeptides were extracted therefrom.

Out of the 96 polypeptides, 26 were expressed in soluble form and achieved higher expression titers than the polypeptide including SC insulin. These 26 fusion polypeptides were selected for the next step. An additional 8 fusion polypeptides, which achieved lower yield but were from peptide categories which were under-represented in the higher-yield polypeptides, were also selected to continue assessment for growth-enhancing activity.

A bioactivity assay was performed for detecting binding of the insulin receptor (IR) by the 34 selected polypeptides, using a PathHunter® kit. Each of the 34 selected polypeptides, as well as SC insulin polypeptide (SEQ ID NO: 10) and human insulin (as a positive control) were respectively added to separate plates of PathHunter U2OS INSRb Bioassay Cells. Following addition of the tested polypeptides, the cells were incubated at room temperature (RT) for 3 hours in the dark. Next, Detection reagent 1 of the PathHunter kit was added to the cells, followed by 15 min incubation at RT in the dark, and then Detection reagent 2 of the PathHunter kit was added, followed by 90 min incubation at RT in the dark. The cell luminescence, resulting from binding of the Insulin receptor by the tested polypeptides, was then detected using a standard luminescence plate reader. Eight of the fusion polypeptides which displayed the highest bioactivity results (i.e., highest activation of the IR) were selected to continue to an in-vitro analysis of growth-promoting activity in cell culture. The selected 8 polypeptides are SEQ ID NO: 11-15, and SEQ ID NO: 17-19. One polypeptide (SEQ ID NO: 16) which produced pronouncedly poor results in the bioactivity assay was selected as a negative control.

### Example 2: Evaluation of Growth Enhancing Effect of Selected Polypeptides in 2D Cell Culture

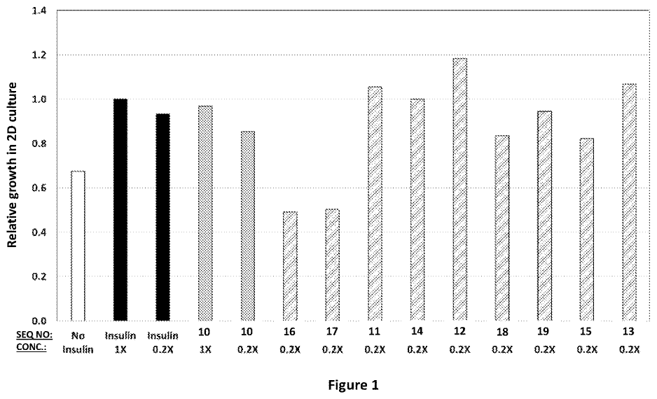

Following the PathHunter bioactivity assay, a cell growth analysis was performed to test the 8 selected fusion polypeptides. Bovine embryonic stem cells (bESCs) were grown in 2D cell culture (i.e., as monolayers on adherent cell plates) with media containing, respectively: recombinant human insulin (Biogems, Cat #10-365) at a baseline concentration of 2.5 μg/ml (captioned “1×”) or at a 5-fold reduced concentration (captioned “0.2×”); a SC-insulin fusion polypeptide (SEQ ID NO: 10) at the baseline weight concentration or at the 5-fold reduced concentration; or the 9 different polypeptides from the selected fusion polypeptides at the 5-fold reduced concentration relative to the baseline concentration. The baseline insulin concentration was selected from the lower end of the range of insulin concentrations which have been reported in the art for mammalian cell-culture. The cells were grown for the duration of 2 passages, each passage for the duration of 3 or 4 days, at the end of which the number of viable cells from each condition was assessed using Trypan Blue exclusion cell count method. The results following two passages are presented in FIG. 1, the tested polypeptides and the respective concentrations are indicated in the graph.

As can be seen in the results, a comparable cell growth was obtained in 2D cell culture when the concentration of WT insulin or SC insulin polypeptide were reduced 5-fold, relative to the baseline concentration. The growth level of the cells in the presence of the negative control (SEQ ID NO: 16) was comparable to the cell growth with no insulin. Regarding the selected fusion polypeptides comprising SEQ ID NO: 11-15 and SEQ ID NO: 17-19, the growth levels in the presence of SEQ ID NO: 11-15, 18 and 19 were comparable to growth levels in the presence of insulin. SEQ ID NO: 17 produced results comparable to the negative control.

It is noted that the comparison of the concentrations of the fusion polypeptides to the concentration of insulin is calculated and presented in weight concentrations of μg/ml. However, the MW of the fusion polypeptides is about 2.5 to 3.5 times higher than the MW of WT insulin, such that, at corresponding weight concentrations, the molar concentration of the fusion polypeptides in the cell medium is about 2.5 to 3.5 times lower than the molar concentrations of the WT insulin in the cell medium. The comparable growth levels, produced by the fusion polypeptides compared to the WT insulin are therefore especially surprising, when the relative molar concentrations are considered.

### Example 3: Evaluation of Growth Enhancing Effect of Selected Polypeptides in Suspension Culture

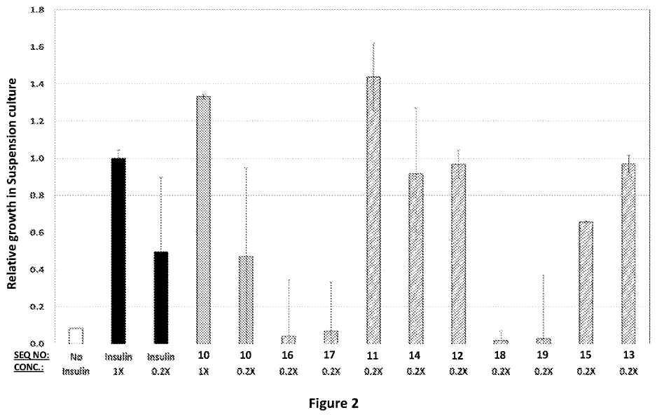

Following the cultivation and growth analysis of cell populations in 2D cell culture described in Example 2, the growth rate of the bESCs was then tested in suspension culture, which is also a commonly used cell-growth method, for example in cultured meat production. bESC populations were grown for 4 days in suspension culture (i.e., in low-adherent 6-well plates subjected to shaking) with media containing, respectively: WT insulin as in Example 2, at a baseline weight concentration of 2.5 μg/ml (captioned “1×”) or at a 5-fold reduced concentration (captioned “0.2×”); a fusion polypeptide including the SC-insulin (SEQ ID NO: 10) at the baseline concentration or at the 5-fold reduced concentration; or respective polypeptides from the selected fusion polypeptides (and the negative control) at a 5-fold reduced weight concentration. The cells were grown for the duration of 4 days, at the end of which the cell aggregates were disaggregated, and the number of cells in each well was assessed using trypan blue exclusion test to assess viable cell number. The results of the cell count are presented in FIG. 2 (mean growth level of two repeats), the identity of the tested polypeptides and the respective concentrations thereof are indicated in the graph.

As can be seen in the results, in suspension culture there were substantial differences between cells grown in the presence of the different concentrations of native insulin, the different concentrations of SC insulin polypeptide, and the different fusion polypeptides. Surprisingly, cell media which contained the fusion polypeptides comprising one of SEQ ID NO: 11, SEQ ID NO: 12, SEQ ID NO: 13, and SEQ ID NO: 14, respectively, achieved cell growth levels substantially higher than those achieved by insulin at the same weight concentrations (5-fold reduction, “0.2×”). SEQ ID NO: 15 also achieved enhanced cell growth, although by a relatively small margin. Notably, SEQ ID NO: 11 achieved cell growth which surpassed even the cell growth achieved by media including insulin at the baseline concentration (1×). SEQ ID NO: 10 (i.e., SC insulin fused to heterologous amino acid sequence SEQ ID NO: 8) achieved higher cell growth than insulin but only at the baseline concentration. Conversely, media which included the polypeptides comprising SEQ ID NO: 16 (negative control), SEQ ID NO: 17, SEQ ID NO: 18, and SEQ ID NO: 19, achieved very low cell growth, comparable to the cell growth in the cell population which was grown with no insulin and no replacement fusion polypeptide.

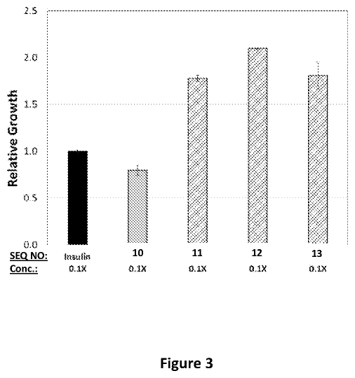

To further explore the cell growth in media containing low concentration of the fusion polypeptides which showed the most pronounced results, cell populations were then grown in suspension culture, as described above, in cell growth media containing WT insulin, SC insulin polypeptide (SEQ ID NO: 10), SEQ ID NO: 11, SEQ ID NO: 12, and SEQ ID NO: 13, respectively, at a 10-fold reduced concentration (caption 0.1×). The cell growth in the presence of WT insulin 0.1× was set as the benchmark growth level. The results are presented in FIG. 3.

Surprisingly, as can be clearly seen in FIG. 3, fusion polypeptides of SEQ ID NO: 11, SEQ ID NO: 12, and SEQ ID NO: 13 each showed substantially elevated cell growth levels relative to the WT insulin, with SEQ ID NO: 12 achieving over 2-fold cell growth.

As mentioned above, it is noted that the comparison of the concentrations of the fusion polypeptides to the concentration of insulin in Examples 2 and 3 above, is calculated and presented in weight concentrations of μg/ml. However, as the MW of the fusion polypeptides is about 2.5 to 3.5 times higher than the MW of WT insulin, the relative molar concentration of the fusion polypeptides is substantially lower than the molar concentration of insulin, which renders the results of enhanced growth even more striking.

Next, the effect on cell growth of additional fusion polypeptides comprising SEQ ID NO: 2-6, and of peptides consisting of SEQ ID NO: 2-6, is tested in an assay similar to the one described in Example 3.

### Example 4: Evaluation of Effect of Selected Peptides on Differentiation

The effect on cell differentiation of additional fusion polypeptides comprising SEQ ID NO: 2-6 and of peptides consisting of SEQ ID NO: 2-6 is tested in a cell differentiation assay, such as a differentiation assay of embryonic fibroblasts into adipocytes or into myocytes. For example, the assay described in International Application Publication WO 2020/230138 A1 (Example 10 therein) for evaluating differentiation of bovine embryonic fibroblasts into myocytes, may be performed, with the peptides or fusion-polypeptides comprising SEQ ID NO: 2-6 being added to the cell differentiation media, optionally replacing at least one component of the media described therein.

### Example 5: Evaluation of Effect of Selected Peptides on Human Cells

The effect on human-derived cell growth, particularly the effect on cell viability and proliferation, of additional fusion polypeptides comprising SEQ ID NO: 2-6 and of peptides consisting of SEQ ID NO: 2-6, is tested using suitable assays.

1-41. (canceled)

42. A growth-enhancing fusion polypeptide, comprising:
a sequence selected from the group consisting of SEQ ID NO: 2, SEQ ID NO: 3, SEQ ID NO: 4, SEQ ID NO: 5, SEQ ID NO: 6, and an analog or derivative thereof; and
a heterologous sequence of 5-100 amino acid residues.

43. The fusion polypeptide according to claim 42, wherein the heterologous sequence comprises at least one moiety selected from: a solubility-enhancing peptide, a carrier polypeptide, and a permeability enhancing moiety, wherein the solubility-enhancing peptide comprises SEQ ID NO: 7, or an analog or derivative thereof.

44. The fusion polypeptide according to claim 42, wherein the heterologous sequence comprises SEQ ID NO: 8, or an analog or derivative thereof.

45. The fusion polypeptide according to claim 42, consisting of a sequence selected from SEQ ID NO: 11, SEQ ID NO: 12, SEQ ID NO: 13, or an analog or derivative thereof.

46. The fusion polypeptide according to claim 42, for use in enhancing cell growth in cell culture or tissue culture, wherein enhancing cell growth comprises enhancing at least one of proliferation, differentiation, and maturation, or for use in replacing insulin as a growth factor in cell culture or tissue culture.

47. The fusion polypeptide for use according to claim 46, wherein the cell culture comprises cells grown in suspension culture.

48. The fusion polypeptide for use according to claim 46, wherein the cell culture is used in the process of producing cultured meat or the tissue culture is further formulated to cultured meat.

49. The fusion polypeptide according to claim 42, for use in enhancing cell growth, wherein the fusion polypeptide promotes cell growth at a low concentration in cell culture relative to insulin concentration in cell culture, wherein the low concentration is low molar concentration, and wherein the low molar concentration is less than 0.33, less than 0.25, less than a 0.15, or less than 0.1 of the insulin molar concentration in cell culture.

50. A polynucleotide encoding a growth-enhancing fusion polypeptide according to claim 42.

51. The polynucleotide according to claim 50, comprising a sequence selected from SEQ ID NO: 21, SEQ ID NO: 22, SEQ ID NO: 23, and an analog or derivative having at least 80% identity to the selected nucleic acid sequence.

52. A cell-culture medium, comprising at least one polypeptide according to claim 42 or at least one peptide selected from the group consisting of SEQ ID NO: 2, SEQ ID NO: 3, SEQ ID NO: 4, and an analog or derivative thereof.

53. The cell-culture medium according to claim 52, wherein the at least one polypeptide or the at least one peptide is present at a weight concentration of about 0.1 μg/ml to 1 μg/ml, or is present at a molar concentration of about 5 nM to 200 nM.

54. The cell-culture medium according to claim 52, wherein the medium is serum-free.

55. A peptide comprising a sequence selected from SEQ ID NO: 2, SEQ ID NO: 3, SEQ ID NO: 4, and an analog or derivative thereof, for use as a growth-enhancing factor.

56. The peptide for use according to claim 55, for enhancing cell growth in a cell culture.

57. A method for culturing cells comprising supplementing a growth medium with at least one polypeptide according to claim 42 or at least one peptide selected from the group consisting of SEQ ID NO: 2, SEQ ID NO: 3, SEQ ID NO: 4, and an analog or derivative thereof, and adding cells to the growth medium.

58. The method according to claim 57, wherein the cells comprise non-human animal-derived cells.

59. The method according to claim 57, wherein culturing the cells is in the process of producing cultured meat.

60. The method according to claim 57, wherein following said supplementing of the growth medium the at least one polypeptide or the at least one peptide is present in the growth medium at a weight concentration of about 0.1 μg/ml to 1 μg/ml, or is present in the growth medium at a molar concentration of about 5 nM to 200 nM.

61. A peptide of up to 100 amino acids, or a fusion polypeptide thereof comprising SEQ ID NO: 4.
